(19) United States
(12) Patent Application Publication
(10) Pub. No.: US 2012/0259265 A1
(43) Pub. Date: Oct. 11, 2012

(54) Regulated Gravity-Based Cerebral Spinal Fluid Drainage Device

(75) Inventors: Sean Salehi, Chicago, IL (US); Mahmound Matthew Salehi, Chicago, IL (US); Jonathan Ferris, Chicago, IL (US)

(73) Assignee: NEUROENTERPRISES, LLC, Chicago, IL (US)

(21) Appl. No.: 13/497,872

(22) PCT Filed: Sep. 17, 2010
(86) PCT No.: PCT/US10/49323

§ 371 Date: Mar. 23, 2012

Related U.S. Application Data
(60) Provisional application No. 61/277,430, filed on Sep. 25, 2009.
(60) Provisional application No. 61/365,718, filed on Jul. 19, 2010.

(51) Int. Cl. A61M 1/00

(52) U.S. Cl. 604/9

(57) ABSTRACT

Portable external gravity-based devices and methods for regulating cerebral spinal fluid drainage from brain, tissue or organs of a patient, and methods for reducing secondary brain injury to a patient by externally regulating the amount of gravity-based cerebral spinal fluid drainage from brain or spine are provided by this invention.

### BACKGROUND OF THE INVENTION

1. Field of the Invention

This invention relates generally to medical devices and methods for externally draining cerebrospinal fluid (CSF) from a brain and a spine. More particularly, the present invention relates to improved medical devices and methods for regulated gravity-based CSF drainage from the brain and the spine without the constant supervision of a caregiver.

2. Description of Related Art

Generally, drainage of CSF fluid is necessary during post-neurosurgical procedures, ENT procedures or spinal procedures when CSF is encountered and where sealing of the dura mater (the sac that contains the brain, the spinal cord and the CSF) at the surgical site is desired. Generally, drainage of CSF is desired via re-routing the CSF from an alternate pathway in the form of an external ventricular drain or lumbar drain). Such neurosurgical or ENT procedures include, for example, skull base surgery, pituitary surgery, traumatic skull base fractures, and sinus surgery (ethmoid, sphenoid and mastoid), as well as spinal procedures. To maintain the neccessary CSF volume and pressure in the spinal canal and to seal the duratomy opening it is often desirable to continuously drain CSF, usually to an external device.

Examples of systems to continuously drain excess CSF to an external device are the Becker System® and the EDM Drainage System® made and sold by Medtronic, PS Medical. Another example of a system to continuously drain excess CSF is shown in U.S. Pat. No. 4,731,056. A further such system is disclosed in U.S. Pat. No. 5,772,625.

Examples of systems for continuously draining excess CSF from the brain to other parts of the body are the Delta® Shunt Assemblies and the CSF-Flow Control Shunt Assemblies made and sold by Medtronic, PS Medical of Goleta, Calif., as disclosed in U.S. Pat. No. 4,560,375.

Currently, CSF drainage control from the brain and spine is limited to the use of a manual valve. Such valves, operated by a caregiver, allow small amounts of CSF drain into a drain bag. Such systems are resistant to automation, because the caregiver must continuously monitor the amounts of CSF that are drained; severe damage can result if too much or too little fluid is drained. Such systems are also inherently variable, being almost completely dependent for their safety and efficacy on the competence of the caregiver and patient-related factors such as coughing or moving.

There are additional drawbacks to the currently-available systems for external drainage of CSF. For example, current systems have no alarm or other warning system. The typical external drainage systems in use today have a manual on/off valve that provides CSF flow control into the collection apparatus. When a patient needs to be moved for short periods of time, for example when being transported for testing or diagnostic work or simply to stand up or go to the bathroom, the valve is turned off by the caregiver to avoid siphoning off too much CSF from a patient's brain and spine. Once the patient's activities have stopped and the patient is settled back into a lying or otherwise stable position, the caregiver reopens the valve or valves to allow the CSF to resume its flow from the brain and the spinal canal. However, in the business of resettling the patient to a physically stable position, the caregiver can easily forget to reopen the valve or valves to allow resumed CSF flow. If the valve(s) are not reopened, the CSF is held in the brain and in the spine, causing intracranial and intraspinal pressure to rise above normal levels. This can lead to brain and spine injury, coma, CSF leak and ultimately death if not caught in time.

Another problem with external drainage systems is how the volume and rate of flow of CSF from a patient's brain and spinal canal are determined. Currently, external drainage systems have a CSF collection apparatus, typically a clear tube marked in increments of cubic centimeters and fractions of centimeters that the caregiver uses to visually determine how much CSF has been collected. The caregiver then has to manually calculate the volume and rate of flow of CSF from that patient during a given time period (typically performed hourly). This can be very difficult to monitor accurately: typically the caregiver is a nurse who is caring for several patients at the same time, making it difficult for the nurse to be present to take measurements precisely on the hour. This leads to inaccuracies in the monitoring and recording of the actual CSF flow rate and collection volume.

The current drainage systems on the market permit CSF drainage but have inherent deficiencies. Accordingly, the inventors have identified a need in the art and practice of medicine to provide a system and a method for portable automated external gravity-based regulation of CSF drainage.

### SUMMARY OF THE INVENTION

It is against the above background that the present invention provides certain advantages and advancements over the prior art.

Although the invention as set forth herein is not limited to specific advantages or functionalities, it is noted that in several embodiments the invention provides devices and methods for externally regulating the amount of cerebral spinal fluid drainage.

Disclosed herein are various embodiments of a device for regulating cerebral spinal fluid drainage amounts from brain, spine, tissue or organs of a patient comprising:

a user interface module comprising logic, an input interface, a communication system to communicate a signal from a sensor mechanism of a fluid-handling module, a first component of a physical connection, and a first component of an electrical connection, and

the fluid-handling module comprising a sensor mechanism, an energy source, a cerebral spinal fluid tubing pathway, a valve mechanism, a second component of physical connection and a second component of the electrical connection, wherein the user interface module and the fluid-handling module are operatively connected through the first component and the second component of the electrical connection,

wherein the device is portable external gravity-based device.

In some embodiments, the user interface module and the fluid-handling module are separable.

Also disclosed herein is a fluid-handling module comprising a housing, a sensor mechanism, an energy source, a cerebral spinal fluid tubing pathway, a valve mechanism, and a component of an electrical connection, wherein the energy source is a battery, is rechargeable or is a capacitor, wherein the battery is selected from the group comprising an alkaline battery, a lithium battery, and a NiMH battery.

Also disclosed herein is a user interface module, comprising a housing, logic, input interface, a communication system, and a component of an electrical connection.

Also disclosed herein is a device for regulating cerebral spinal fluid drainage amounts from brain, spine, tissue or organs of a patient comprising a single module, further comprising a housing, logic, input interface, a sensor mechanism, a communication system to communicate a signal from the sensor mechanism, an energy source, a cerebral spinal fluid tubing pathway, and a valve mechanism, wherein the device is portable external gravity-based device.

As described herein, the organs can include, but are not limited to organs of the body such as the liver, kidneys, the heart, and the bladder.

In some embodiments, the first component of the electrical connection of a device of the invention is a male connector and wherein the second component of the electrical connection of a device of the invention is a female connector, wherein the electrical connection is between the male connector and the female connector.

In further embodiments, the fluid-handling module of a device of the invention or, in alternative embodiments, the fluid-handling module of the invention, or in further alternative embodiments, a single module of a device of the invention comprises an energy source for powering itself and the user interface module, wherein the energy source is a battery, is rechargeable or is a capacitor, wherein the battery is an alkaline battery, a lithium battery or a NiMH battery.

In some embodiments, the user interface module of a device of the invention, or in alternative embodiments, the user interface module of the invention comprises an energy source for powering itself and the fluid-handling module, wherein the energy source is a battery, is rechargeable or is a capacitor, wherein the battery is an alkaline battery, a lithium battery or a NiMH battery.

In some embodiments, a device of the invention has an on/off switch. In some embodiments, a device of the invention does not have an on/off switch, wherein the device is automatically powered on when the user interface module and the fluid-handling module are connected, and wherein the device is automatically powered off when the user interface module and the fluid-handling module are disconnected.

In further embodiments, the fluid-handling module of a device of the invention or, in alternative embodiments, the fluid-handling module of the invention, or in further alternative embodiments, a single module of a device of the invention comprises a housing made of, for example, but not limited to thermoplastic, having an inlet opening and an outlet opening, wherein the inlet opening is adapted to be coupled to a shunt line and the outlet opening is adapted to be coupled to a drainage line, wherein the fluid-handling module is coupled to the shunt line and the drainage line via Luer locks or other medical connectors.

In some embodiments, the fluid-handling module of a device of the invention or, in alternative embodiments, the fluid-handling module of the invention, or in further alternative embodiments, of a single module of a device of the invention is sterile and disposable, wherein the fluid-handling module is discarded after each patient's use.

In some embodiments, the valve mechanism of the fluid-handling module of a device of the invention or, in alternative embodiments, of the fluid-handling module of the invention, or in further alternative embodiments, of a single module of a device of the invention can be, but is not limited to, a pinch solenoid-operated valve mechanism that fills and periodically drains a small flexible compartment or an internal bag, further comprising a first valve, wherein the first valve allows drainage of the cerebral spinal fluid from a shunt line into the flexible compartment or the internal bag, and a second valve, wherein the second valve allows drainage of the cerebral spinal fluid from the flexible compartment or the internal bag into an external drain bag. The small flexible compartment or the internal bag holds a pre-determined amount of CSF and the device controls the rate the small flexible compartment or the internal bag is filled and drained, wherein the device is capable of emptying the user-determined amount of CSF each hour.

In some embodiments, the sensor mechanism of the fluid-handling module of a device of the invention or, in alternative embodiments, of the fluid-handling module of the invention, or in further alternative embodiments, of a single module of a device of the invention further comprises a first sensor and a second sensor, wherein the first sensor is located on the flexible compartment, or the internal bag, wherein the first sensor comprise a pair of electrodes used to sense a change in the amount of fluid in the flexible compartment or the internal bag, and the second sensor is located on the battery, wherein the first sensor of the sensor mechanism detects when the cerebral spinal fluid tubing pathway is clogged and sends a signal to the user interface module of a device of the invention if the change in the amount of fluid in the internal bag is lower than a user predetermined value, wherein the second sensor of the sensor mechanism detects a low battery condition and sends a signal to the user interface module of a device of the invention.

In some embodiments, the housing of the fluid-handling module of a device of the invention or, in alternative embodiments, of the fluid-handling module of the invention, or in further alternative embodiments, of a single module of a device of the invention comprises an adjustable bracket, a fluid-handling module-securing screw, and two bracket-securing screws, wherein the bracket in a vertical configuration is capable of attaching to a support member, wherein the support member can be, but is not limited to, an IV pole, by adjusting the bracket placement, wherein the bracket in a horizontal configuration is capable of attaching to a support member, wherein the support member can be, but is not limited to, a bed rail, by adjusting the bracket placement.

In some embodiments, the user interface module of a device of the invention, or in alternative embodiments, the user interface module of the invention, or in further alternative embodiments, a single module of a device of the invention is non-sterile and reusable.

In further embodiments, the logic of the user interface module of a device of the invention, or in alternative embodiments, of the user interface module of the invention, or in further alternative embodiments, of a single module of a device of the invention is configured to receive, display or process a signal from the sensor mechanism of the fluid-handling module or the input interface, or to cause an action to be taken, to regulate the amount of cerebral spinal fluid drained to no more than 20 cubic centimeters per hour or to compare the current flow rate limit parameter received in the signal from the sensor mechanism of the fluid-handling module with the user-predetermined flow rate range.

In some embodiments, the logic of the user interface module of a device of the invention, or in alternative embodiments, of the user interface module of the invention, or in further alternative embodiments, of a single module of a device of the invention comprises a microprocessor, wherein the microprocessor further comprises memory, wherein the memory includes instructions that, when executed by the microprocessor causes the microprocessor to receive, display, store or process the signal from the sensor mechanism of the fluid-handling module or the input interface, or to cause action to be taken, wherein the signal from the sensor mechanism of the fluid-handling module comprises a current flow rate limit parameter, a clogged cerebral spinal fluid tubing pathway signal or a low battery condition signal.

In further embodiments, the logic of the user interface module of a device of the invention, or in alternative embodiments, of the user interface module of the invention, or in further alternative embodiments, of a single module of a device of the invention comprises random access memory, wherein the random access memory includes instructions that, when executed causes the logic to receive, display, store or process the signal from the sensor mechanism of the fluid-handling module or the input interface, or to cause an action to be taken, wherein the signal from the sensor mechanism of the fluid-handling module comprises a current flow rate limit parameter, a clogged cerebral spinal fluid tubing pathway signal or a low battery condition signal.

In some embodiments, the input interface of the user interface module of a device of the invention, or in alternative embodiments, of the user interface module of the invention, or in further alternative embodiments, of a single module of a device of the invention is used to set a fill/drain timing parameter to increase or decrease the amount of cerebral spinal fluid drained, a user-predetermined flow rate range or a time of day.

In some embodiments, the logic of the user interface module of a device of the invention, or in alternative embodiments, of the user interface module of the invention, or in further alternative embodiments, of a single module of a device of the invention comprises an alarm, wherein the logic activates and sounds the alarm to alert a user when the current flow rate limit parameter received in the signal from the sensor mechanism of the fluid-handling module is outside of the user-predetermined flow rate range, to alert a user when a clogged cerebral spinal fluid tubing pathway signal is received from a clog sensor in the fluid-handling module, wherein the clog sensor is a capacitance sensor, a weight sensor, a flow sensor, a strain gauge sensor, a potentiometric sensor, an optical sensor or a magnetic sensor, or to alert a user when a low battery condition signal is received from the fluid-handling module.

In further embodiments, the communication system of the user interface module of a device of the invention, or in alternative embodiments, of the user interface module of the invention, or in further alternative embodiments, of a single module of a device of the invention comprises a display system that communicates the signal to a user, wherein the display system further comprises a display screen that displays parameter information to the user, wherein the parameter information displayed to the user can generally include but is not limited to the current flow rate limit, an amount drained over a time interval, which can be, for example, but not limited to, last hour, since midnight of the previous day, and during the previous day.

In some embodiments, the display system of the user interface module of a device of the invention, or in alternative embodiments, of the user interface module of the invention, or in further alternative embodiments, of a single module of a device of the invention further comprises a light-emitting diode that flashes periodically to indicate that the system is functioning normally, wherein the light-emitting diode is, but not limited to, green light-emitting diode.

In some embodiments, the display system of the user interface module of a device of the invention, or in alternative embodiments, of the user interface module of the invention, or in further alternative embodiments, of a single module of a device of the invention further comprises a light-emitting diode that flashes continuously to indicate that the logic has received a clogged cerebral spinal fluid tubing pathway signal from the fluid-handling module, or a signal from the fluid-handling module that the current flow rate limit parameter is outside of the user-predetermined flow rate range, or a low battery condition signal from the fluid-handling module, wherein the light-emitting diode is, but not limited to, red light-emitting diode.

In some embodiments the input interface of the user interface module of a device of the invention, or in alternative embodiments, of the user interface module of the invention, or in further alternative embodiments, of a single module of a device of the invention further comprises a member, which can be, but is not limited to, a button to increase the cerebral spinal fluid drainage limit, a member, which can be, but is not limited to, a button to decrease the cerebral spinal fluid drainage limit, and a member, which can be, but is not limited to, a button to make menu selections, wherein the button to increase the cerebral spinal fluid drainage limit can be an “up” button, wherein the button to decrease the cerebral spinal fluid drainage limit can be a “down” button, and wherein the button to make menu selections can be a “select” button.

In some embodiments, the user interface module of a device of the invention, or in alternative embodiments, of the user interface module of the invention, or in further alternative embodiments, of a single module of a device of the invention further comprises electronics to control the logic, the communication system, the input interface, and the fluid-handling module.

Disclosed herein is also a method of externally regulating gravity-based cerebral spinal fluid drainage from brain, spine, tissue or organs of a patient, comprising the steps of:

positioning the fluid-handling module of a device of the invention, or in alternative embodiments, a single module of a device of the invention at least six inches below the patient's hip to ensure proper outflow through the module by gravity;

attaching the fluid-handling module of a device of the invention, or in alternative embodiments, a single module of a device of the invention to a support member;

connecting the fluid-handling module of a device of the invention to the user interface module, and thereafter powering the user interface module, or in alternative embodiments, powering a single module of a device of the invention by, for example, using an on/off switch;

flushing the cerebral spinal fluid tubing pathway of the fluid-handling module of a device of the invention, or in alternative embodiments, of a single module of a device of the invention the device with 5 cc saline solution using an external syringe the first time the device is being used in a patient;

coupling the fluid-handling module of a device of the invention, or in alternative embodiments, a single module of a device of the invention to the shunt line through the inlet opening and to the drain line through the outlet opening; and

using the logic of the device to set a fill/drain timing schedule to increase or decrease the amount of cerebral spinal fluid drainage required, a volume of CSF drained, or time of day setting or to control the device by setting the range for the cerebral spinal fluid drainage required,

wherein the method is external gravity-based method.

Also disclosed herein is a method of externally regulating gravity-based cerebral spinal fluid drainage from brain, spine, tissue or organs of a patient, comprising the steps of:

positioning the fluid-handling module of a device of the invention at least six inches below the patient's hip to ensure proper outflow through the module by gravity;

attaching the fluid-handling module of the device to a support member;

connecting the fluid-handling module of the device to the user interface module, and

thereafter powering the user interface module;

flushing the cerebral spinal fluid tubing pathway of the device with 5 cc saline solution using an external syringe the first time the device is being used in a patient;

coupling the fluid-handling module of the device to the shunt line through the inlet opening and to the drain line through the outlet opening; and

using the logic of the device to set a fill/drain timing schedule to increase or decrease the amount of cerebral spinal fluid drainage required, a volume of CSF drained, or time of day setting or to control the device by setting the range for the cerebral spinal fluid drainage required,

wherein the method is external gravity-based method.

In some embodiments, a method of externally regulating gravity-based cerebral spinal fluid drainage from brain, spine, tissue or organs of a patient, further comprises the step of controlling the device by setting the range for the cerebral spinal fluid drainage required. In some embodiments, the method further comprises flushing the cerebral spinal fluid tubing pathway of the device with 5 cc saline solution using an external syringe the first time the device is being used in a patient.

In further embodiments, the gravity-based cerebral spinal fluid drainage from brain, spine, tissue or organs of a patient is continuous, for example, but not limited to the gravity-based cerebral spinal fluid drainage three days.

In some embodiments, a method of externally regulating gravity-based cerebral spinal fluid drainage from brain, spine, tissue or organs of a patient, further comprises the step of determining if the continuous drainage of CSF for 3 days results in clinical improvement in patients with suspected Normal Pressure Hydrocephalus (NPH).

In some embodiments, a method of externally regulating gravity-based cerebral spinal fluid drainage from brain, spine, tissue or organs of a patient, further comprises the step of reducing secondary brain injury of a patient by externally regulating the amount of gravity-based cerebral spinal fluid drainage from brain or spine of a patient to prevent subdural hematoma.

In some embodiments, a device of the invention regulates the amount of gravity-based cerebral spinal fluid drainage from the ventricular system of the brain or the intrathecal space of the spinal canal.

Disclosed herein is also a method for improving post-operative outcomes in a patient following a neurosurgical or ENT procedure using a device of the invention, wherein the post neurosurgical or the ENT procedure are triggered by the CSF encounter and wherein sealing of a dura mater at a surgical site via re-routing of the CSF from an alternate pathway in the form of an external ventricular drain or the lumbar drain, wherein the dura mater is a sac which contains the brain, spinal cord and CSF, comprising the steps of:

positioning the fluid-handling module of a device of the invention, or in alternative embodiments, a single module of a device of the invention at least six inches below the patient's hip to ensure proper outflow through the module by gravity;

attaching the fluid-handling module of a device of the invention, or in alternative embodiments, a single module of a device of the invention to a support member;

connecting the fluid-handling module of a device of the invention to the user interface module, and thereafter powering the user interface module, or in alternative embodiments, powering a single module of a device of the invention by, for example, using an on/off switch;

flushing the cerebral spinal fluid tubing pathway of the fluid-handling module of a device of the invention, or in alternative embodiments, of a single module of a device of the invention the device with 5 cc saline solution using an external syringe the first time the device is being used in a patient;

coupling the fluid-handling module of a device of the invention, or in alternative embodiments, a single module of a device of the invention to the shunt line through the inlet opening and to the drain line through the outlet opening; and

using the logic of the device to set a fill/drain timing schedule to increase or decrease the amount of cerebral spinal fluid drainage required, a volume of CSF drained, or time of day setting or to control the device by setting the range for the cerebral spinal fluid drainage required,

wherein the method is external gravity-based method.

A method for improving post-operative outcomes in a patient following a neurosurgical or ENT procedure using a device according to claim 1, wherein the post neurosurgical or the ENT procedure are triggered by the CSF encounter and wherein sealing of a dura mater at a surgical site via re-routing of the CSF from an alternate pathway in the form of an external ventricular drain or the lumbar drain, wherein the dura mater is a sac which contains the brain, spinal cord and CSF, comprising the steps of:

positioning the fluid-handling module of a device of the invention at least six inches below the patient's hip to ensure proper outflow through the module by gravity;

attaching the fluid-handling module of the device to a support member;

connecting the fluid-handling module of the device to the user interface module, and thereafter powering the user interface module;

flushing the cerebral spinal fluid tubing pathway of the device with 5 cc saline solution using an external syringe the first time the device is being used in a patient;

coupling the fluid-handling module of the device disclosed herein to the shunt line through the inlet opening and to the drain line through the outlet opening; and

using the logic of the device to set a fill/drain timing schedule to increase or decrease the amount of cerebral spinal fluid drainage required, a volume of CSF drained, or time of day setting or to control the device by setting the range for the cerebral spinal fluid drainage required,

wherein the method is external gravity-based method.

In some embodiments, the post neurosurgical or the ENT procedure is a skull base surgery, a pituitary surgery, a traumatic skull base fracture, a post spinal surgery, a sinus surgery, wherein the sinus surgery is an ethmoid, sphenoid or mastoid.

In some embodiments, a device of the invention further comprises a pressure transducer attached to the catheter exiting from the ventricle before the inlet opening of the fluid-handling module and is adapted for measuring intracranial pressure by the transducer, wherein the transducer communicates pressure information to the user interface module, or in alternative embodiments, to a single module of a device of the invention via logic, wherein the pressure information is displayed on the display screen, wherein the logic sets, activates and sounds the alarm to alert a user when the pressure is above the user-predetermined pressure range.

In further embodiments, the user interface module of a device of the invention, or in alternative embodiments, of a single module of a device of the invention contains a USB port or a wireless transmitter.

Also disclosed herein is a method for uploading parameter information from a device of the invention into an electronic medical record (EMR) comprising the steps of: establishing a USB or a wireless connection between a device of the invention and an external computer system, wherein the external computer system contains the electronic medical record; and uploading parameter information from the device into the electronic medical record of the external computer system.

In further embodiments, the parameter information can generally include but is not limited to the current flow rate limit, an amount drained last hour, an amount drained since midnight, and an amount drained over a time interval, for example, but not limited to an amount drained the previous day, wherein the parameter information can generally further include but is not limited to includes a signal received by the logic, wherein the signal is a clogged cerebral spinal fluid tubing pathway signal from the fluid-handling module, or in alternative embodiments, from a single module of a device of the invention, the signal from the fluid-handling module, or in alternative embodiments, from a single module of a device of the invention that the current flow rate limit parameter is outside of the user-predetermined flow rate range or the low battery condition signal from the fluid-handling module, or in alternative embodiments, from a single module of a device of the invention.

The use of a device of the invention, wherein the device is positioned at least 6 inches below the patient's hip to ensure proper outflow through the device, wherein the device is positioned generally upright for generally vertical downward flow of cerebral spinal fluid therethrough, wherein an inflow opening at the upper end of the device is adapted to be coupled to a shunt line and an outflow opening at the lower end of the device is adapted to be coupled to the drainage line.

The use of a device of the invention in a post neurosurgical or an ENT procedures wherein the post neurosurgical or the ENT procedure are triggered by the CSF encounter and wherein sealing of a dura mater at a surgical site via re-routing of the CSF from an alternate pathway in the form of an external ventricular drain or the lumbar drain is desired, wherein the dura mater is a sac which contains the brain, spinal cord and CSF.

Also disclosed herein is a fluid-handling module comprising a sensor mechanism, an energy source, a cerebral spinal fluid tubing pathway, a valve mechanism, and a component of an electrical connection.

The fluid-handling module, wherein the energy source is a battery, is rechargeable or is a capacitor, wherein the battery is selected from the group comprising an alkaline battery, a lithium battery, and a NiMH battery.

The fluid-handling module, comprising a thermoplastic housing having an inlet opening and an outlet opening, wherein the inlet opening is adapted to be coupled to a shunt line and the outlet opening is adapted to be coupled to a drainage line, wherein the fluid-handling module is coupled to the shunt line and the drainage line via Luer locks or similar medical connectors.

The fluid-handling module, wherein the fluid-handling module is sterile and disposable, wherein the fluid-handling module is discarded after each patient's use.

The fluid-handling module, wherein the valve mechanism can be, but is not limited to, a pinch solenoid-operated valve mechanism that fills and drains a small flexible compartment or an internal bag, periodically, further comprising a first valve, wherein the first valve allows drainage of the cerebral spinal fluid from a shunt line into the flexible compartment or the internal bag, and a second valve, wherein the second valve allows drainage of the cerebral spinal fluid from the flexible compartment or the internal bag into an external drain bag.

The fluid-handling module, wherein small flexible compartment or the internal bag holds a pre-determined amount of CSF and the device controls the rate the small flexible compartment or the internal bag is filled and drained, wherein the device is capable of emptying the user-determined amount of CSF each hour.

The sensor mechanism of the fluid-handling module, wherein the sensor mechanism further comprises a first sensor and a second sensor, wherein the first sensor is located on the flexible compartment, or the internal bag, wherein the first sensor comprise a pair of electrodes used to sense a change in the amount of fluid in the flexible compartment or the internal bag, and the second sensor is located on the battery, wherein the first sensor of the sensor mechanism detects when the cerebral spinal fluid tubing pathway is clogged and sends a signal to the user interface module of a device of the invention if the change in the amount of fluid in the internal bag is lower than a user predetermined value, wherein the second sensor of the sensor mechanism detects a low battery condition and sends a signal to the user interface module of a device of the invention.

The fluid-handling module, wherein the housing of the fluid-handling module comprises an adjustable bracket, a fluid-handling module-securing screw, and two bracket-securing screws, wherein the bracket in a vertical configuration is capable of attaching to a support member, wherein the support member can be, but is not limited to, an IV pole, by adjusting the bracket placement, wherein the bracket in a horizontal configuration is capable of attaching to a support member, wherein the support member can be, but is not limited to, a bed rail, by adjusting the bracket placement.

The fluid-handling module is sterile and disposable, wherein the fluid-handling module is discarded after each patient's use.

Also disclosed herein is a user interface module comprising a thermoplastic housing, logic, input interface, a communication system, and a component of an electrical connection.

The user interface module, wherein the user interface module is non-sterile and reusable.

The user interface module, where the logic is configured to receive, display or process a signal from the sensor mechanism of the fluid-handling module or the input interface, or to cause an action to be taken, to regulate the amount of cerebral spinal fluid drained to no more than 20 cubic centimeters per hour or to compare the current flow rate limit parameter received in the signal from the sensor mechanism of the fluid-handling module with the user-predetermined flow rate range.

The user interface module, wherein the logic comprises a microprocessor, wherein the microprocessor further comprises memory, wherein the memory includes instructions that, when executed by the microprocessor causes the microprocessor to receive, display, store or process the signal, or to cause action to be taken.

The user interface module, wherein the logic comprises random access memory, wherein the random access memory includes instructions that, when executed causes the logic to receive, display, store or process the signal, or to cause action to be taken.

The user interface module, wherein the input interface is used to set a fill/drain timing parameter to increase or decrease the amount of cerebral spinal fluid drained, a user-predetermined flow rate range or a time of day.

The user interface module, wherein the logic comprises an alarm, wherein the logic activates and sounds the alarm to alert a user when the current flow rate limit parameter received in the signal from the sensor mechanism of the fluid-handling module is outside of the user-predetermined flow rate range, to alert a user when a clogged cerebral spinal fluid tubing pathway signal is received from a clog sensor in the fluid-handling module, wherein the clog sensor is a capacitance sensor, a weight sensor, a flow sensor, a strain gauge sensor, a potentiometric sensor, an optical sensor or a magnetic sensor, or to alert a user when a low battery condition signal is received from the fluid-handling module.

The user interface module, wherein the communication system comprises a display system that communicates the signal to a user, wherein the display system further comprises a display screen that displays parameter information to the user, wherein the parameter information displayed to the user can generally include but is not limited to the current flow rate limit, an amount drained over a time interval, which can be, for example, but not limited to, last hour, since midnight of the previous day, and during the previous day.

The user interface module, wherein the display system further comprises a light-emitting diode that flashes periodically to indicate that the system is functioning normally, wherein the light-emitting diode is, but not limited to, green light-emitting diode.

The user interface module, wherein the display system further comprises a light-emitting diode that flashes continuously to indicate that the logic has received a clogged cerebral spinal fluid tubing pathway signal from the fluid-handling module, or a signal from the fluid-handling module that the current flow rate limit parameter is outside of the user-predetermined flow rate range, or a low battery condition signal from the fluid-handling module, wherein the light-emitting diode is, but not limited to, red light-emitting diode.

The user interface module, wherein the input interface further comprises a member, which can be, but is not limited to, a button to increase the cerebral spinal fluid drainage limit, a member, which can be, but is not limited to, a button to decrease the cerebral spinal fluid drainage limit, and a member, which can be, but is not limited to, a button to make menu selections, wherein the button to increase the cerebral spinal fluid drainage limit can be an “up” button, wherein the button to decrease the cerebral spinal fluid drainage limit can be a “down” button, and wherein the button to make menu selections can be a “select” button.

The user interface module, wherein the user interface module further comprises electronics to control the logic, the communication system, the input interface, and the fluid-handling module.

Also disclosed herein is a device for regulating cerebral spinal fluid drainage amounts from brain, spine, tissue or organs of a patient comprising a single module, further comprising a housing, logic, input interface, a sensor mechanism, a communication system to communicate a signal from the sensor mechanism, an energy source, a cerebral spinal fluid tubing pathway, and a valve mechanism, wherein the device is portable external gravity-based device.

The device comprising the single module, wherein the device contains an energy source for powering itself, wherein the energy source is a battery, is rechargeable or is a capacitor, wherein the battery is an alkaline battery, a lithium battery or a NiMH battery.

The device comprising the single module, wherein the device has an on/off switch.

The device comprising the single module, wherein the device comprises a thermoplastic housing having an inlet opening and an outlet opening, wherein the inlet opening is adapted to be coupled to a shunt line and the outlet opening is adapted to be coupled to a drainage line, wherein the fluid-handling module is coupled to the shunt line and the drainage line via Luer locks or similar medical connectors.

The device comprising the single module, wherein the device is sterile and disposable, wherein the device is discarded after each patient's use.

The valve mechanism of the device comprising the single module, wherein the valve mechanism can be, but is not limited to, a pinch solenoid-operated valve mechanism that fills and periodically drains a small flexible compartment or an internal bag, further comprising a first valve, wherein the first valve allows drainage of the cerebral spinal fluid from a shunt line into the flexible compartment or the internal bag, and a second valve, wherein the second valve allows drainage of the cerebral spinal fluid from the flexible compartment or the internal bag into an external drain bag.

The device comprising the single module, wherein the small flexible compartment or the internal bag holds a user-determined amount of the cerebral spinal fluid and enables the device to empty the user-determined amount of the cerebral spinal fluid per hour.

The sensor mechanism of the device comprising the single module, wherein the sensor mechanism further comprises a first sensor and a second sensor, wherein the first sensor is located on the flexible compartment, or the internal bag, wherein the first sensor comprise a pair of electrodes used to sense a change in the amount of fluid in the internal bag, and the second sensor is located on the battery, wherein the first sensor of the sensor mechanism detects if the cerebral spinal fluid tubing pathway is clogged and sends a signal to the communication system if the change in the amount of fluid in the internal bag is lower then the user predetermined value, wherein the second sensor of the sensor mechanism detects a low battery condition and sends a signal to the communication system.

The device comprising the single module, wherein the housing of the device comprises an adjustable bracket, a fluid-handling module-securing screw, and two bracket-securing screws, wherein the bracket in a vertical configuration is capable of attaching to a support member, wherein the support member can be, but is not limited to, an IV pole, by adjusting the bracket placement, wherein the bracket in a horizontal configuration is capable of attaching to a support member, wherein the support member can be, but is not limited to, a bed rail, by adjusting the bracket placement.

The device comprising the single module, wherein the device is non-sterile and reusable.

The device comprising the single module, where the logic is further configured to receive, display or process a signal from the sensor mechanism, or to cause action to be taken.

The device comprising the single module, wherein the logic comprises a microprocessor, wherein the microprocessor further comprises memory, wherein the memory includes instructions that, when executed by the microprocessor causes the microprocessor to receive, display, store or process the signal from the sensor mechanism, or to cause action to be taken, wherein the signal from the sensor mechanism comprises a current flow rate limit parameter, a clogged cerebral spinal fluid tubing pathway signal or a low battery condition signal.

The device comprising the single module, wherein the logic comprises random access memory, wherein the random access memory includes instructions that, when executed causes the logic to receive, display, store or process the signal from the sensor mechanism, or to cause action to be taken, wherein the signal from the sensor mechanism comprises a current flow rate limit parameter, a clogged cerebral spinal fluid tubing pathway signal or a low battery condition signal.

The device comprising the single module, wherein the logic is further configured to regulate the amount of cerebral spinal fluid drained to no more than 20 cubic centimeters per hour, in whole number increments, wherein the logic is further configured to compare the current flow rate limit parameter received in the signal from the sensor mechanism with the user-predetermined flow rate range.

The device comprising the single module, wherein the input interface is used to set a fill/drain timing parameter to increase or decrease the amount of cerebral spinal fluid drained, a user-predetermined flow rate range or a time of day.

The device comprising the single module, wherein the logic comprises an alarm, wherein the logic activates and sounds the alarm to alert a user when the current flow rate limit parameter received in the signal from the sensor mechanism is outside of the user-predetermined flow rate range, to alert a user when a clogged cerebral spinal fluid tubing pathway signal is received from the first sensor in the sensor mechanism, wherein the first sensor is a capacitance sensor, weight sensor, flow sensor, strain gauge sensor, potentiometric sensor, optical sensor or magnetic sensor or to alert a user when a low battery condition signal is received from the second sensor of the sensor mechanism.

The device comprising the single module, wherein the communication system is a display system that communicates the signal to a user, wherein the display system further comprises a display screen that displays parameter information to the user, wherein the parameter information displayed to the user can generally include but is not limited to the current flow rate limit, an amount drained over a time interval, which can be, for example, but not limited to, last hour, since midnight of the previous day, and during the previous day.

The device comprising the single module, wherein the display system further comprises a light-emitting diode that flashes periodically to indicate that the system is functioning normally, wherein the light-emitting diode is, but not limited to, green light-emitting diode.

The device comprising the single module, wherein the display system further comprises a light-emitting diode that flashes continuously to indicate that the logic has received a clogged cerebral spinal fluid tubing pathway signal from the fluid-handling module, or a signal from the fluid-handling module that the current flow rate limit parameter is outside of the user-predetermined flow rate range, or a low battery condition signal from the fluid-handling module, wherein the light-emitting diode is, but not limited to, red light-emitting diode.

The device comprising the single module, wherein the input interface further comprises a member, which can be, but is not limited to, a button to increase the cerebral spinal fluid drainage limit, a member, which can be, but is not limited to, a button to decrease the cerebral spinal fluid drainage limit, and a member, which can be, but is not limited to, a button to make menu selections, wherein the button to increase the cerebral spinal fluid drainage limit can be an “up” button, wherein the button to decrease the cerebral spinal fluid drainage limit can be a “down” button, and wherein the button to make menu selections can be a “select” button.

The device comprising the single module, further comprising electronics to control the logic, the communication system, and the input interface.

Also disclosed herein is a method of externally regulating the amount for gravity-based cerebral spinal fluid drainage from brain, spine, tissue or organs of a patient, comprising the steps of:

positioning the fluid-handling module of a device comprising a single module at least six inches below the patient's hip to ensure proper outflow through the module by gravity;

attaching the fluid-handling module of the device to a support member;

connecting the fluid-handling module of the device to the user interface module, and thereafter powering the user interface module;

flushing the cerebral spinal fluid tubing pathway of the device with 5 cc saline solution using an external syringe the first time the device is being used in a patient;

coupling the fluid-handling module of the device to the shunt line through the inlet opening and to the drain line through the outlet opening; and

using the logic of the device to set a fill/drain timing schedule to increase or decrease the amount of cerebral spinal fluid drainage required, a volume of CSF drained, or time of day setting or to control the device by setting the range for the cerebral spinal fluid drainage required,

wherein the method is external gravity-based method.

The method of externally regulating the amount for gravity-based cerebral spinal fluid drainage from brain, spine, tissue or organs of a patient, further comprising avoiding reinfusion of the drained CSF into the brain or spine and preventing introduction of infection or raising of the intracranial or intra spinal pressure.

The method of externally regulating the amount for gravity-based cerebral spinal fluid drainage from brain, spine, tissue or organs of a patient, further comprising the step of controlling the device by setting the range for the cerebral spinal fluid drainage required.

The method of externally regulating the amount for gravity-based cerebral spinal fluid drainage from brain, spine, tissue or organs of a patient, wherein the method further comprises flushing the cerebral spinal fluid tubing pathway of the device with 5 cc saline solution using an external syringe the first time the device is being used in a patient.

The method of externally regulating the amount for gravity-based cerebral spinal fluid drainage from brain, spine, tissue or organs of a patient, wherein the gravity-based cerebral spinal fluid drainage from brain, spine, tissue or organs of a patient is continuous.

The method of externally regulating the amount for gravity-based cerebral spinal fluid drainage from brain, spine, tissue or organs of a patient, further comprising the step of determining if the continuous drainage of CSF for 3 days results in clinical improvement in patients with suspected Normal Pressure Hydrocephalus (NPH).

The method of externally regulating the amount for gravity-based cerebral spinal fluid drainage from brain, spine, tissue or organs of a patient, further comprising the step of reducing secondary brain injury of a patient by externally regulating the amount of gravity-based cerebral spinal fluid drainage from brain or spine of a patient to prevent subdural hematoma.

The device comprising the single module, wherein the device prevents, reduces or inhibits reinfusion of the drained CSF into the brain or spine and prevents introduction of infection or increasing of the intracranial or intra spinal pressure.

The device comprising the single module, wherein the device regulates the amount of gravity-based cerebral spinal fluid drainage from the ventricular system of the brain or the intrathecal space of the spinal canal.

Also disclosed herein is a method for improving post-operative outcomes in a patient following a neurosurgical or ENT procedure using a device comprising the single module, wherein the post neurosurgical or the ENT procedure are triggered by the CSF encounter and wherein sealing of a dura mater at a surgical site via re-routing of the CSF from an alternate pathway in the form of an external ventricular drain or the lumbar drain, wherein the dura mater is a sac which contains the brain, spinal cord and CSF, comprising the steps of:

positioning the fluid-handling module of a device comprising a single module at least six inches below the patient's hip to ensure proper outflow through the module by gravity;

attaching the fluid-handling module of the device to a support member;

connecting the fluid-handling module of the device to the user interface module, and thereafter powering the user interface module;

flushing the cerebral spinal fluid tubing pathway of the device with 5 cc saline solution using an external syringe the first time the device is being used in a patient;

coupling the fluid-handling module of the device to the shunt line through the inlet opening and to the drain line through the outlet opening; and

using the logic of the device to set a fill/drain timing schedule to increase or decrease the amount of cerebral spinal fluid drainage required, a volume of CSF drained, or time of day setting or to control the device by setting the range for the cerebral spinal fluid drainage requiredThe method for improving post-operative outcomes in a patient following a neurosurgical or ENT procedure using a device comprising the single module, wherein the post neurosurgical or the ENT procedure is a skull base surgery, a pituitary surgery, a traumatic skull base fractures, a post spinal surgery, a sinus surgery, wherein the sinus surgery is a ethmoid, sphenoid or mastoid,

wherein the method is external gravity-based method.

The device comprising the single module, wherein the device further comprises a pressure transducer attached to the catheter exiting from the ventricle before the inlet opening of the device and is adapted for measuring intracranial pressure by the transducer, wherein the transducer communicates pressure information to the user interface module via logic, wherein the pressure information is displayed on the display screen, wherein the logic sets, activates and sounds the alarm to alert a user when the pressure is above the user-predetermined pressure range.

The device comprising the single module, wherein the user interface module contains a USB port or a wireless transmitter.

Also disclosed herein is a method for uploading parameter information from a device comprising the single module into an electronic medical record (EMR) comprising the steps of:

establishing a USB or a wireless connection between the device comprising the single module and an external computer system, wherein the external computer system contains the electronic medical record; and

uploading the parameter information from the device comprising the single module into the electronic medical record of the external computer system.

The method for uploading parameter information from a device comprising the single module, wherein the parameter information can include, but is not limited to, the current flow rate limit, an amount drained last hour, an amount drained since midnight, and an amount drained yesterday, wherein the parameter information further includes a signal received by the logic, wherein the signal is a clogged cerebral spinal fluid tubing pathway signal from the fluid-handling module, the signal from the fluid-handling module that the current flow rate limit parameter is outside of the user-predetermined flow rate range or the low battery condition signal from the fluid-handling module.

The use of the device comprising the single module, wherein the device is positioned at least 6 inches below the patient's hip to ensure proper outflow through the device, wherein the device is positioned generally upright for generally vertical downward flow of cerebral spinal fluid therethrough, wherein an inflow opening at the upper end of the device is adapted to be coupled to a shunt line and an outflow opening at the lower end of the device is adapted to be coupled to the drainage line.

The use of the device comprising the single module in a post neurosurgical or an ENT procedures wherein the post neurosurgical or the ENT procedure are triggered by the CSF encounter and wherein sealing of a dura mater at a surgical site via re-routing of the CSF from an alternate pathway in the form of an external ventricular drain or the lumbar drain is desired, wherein the dura mater is a sac which contains the brain, spinal cord and CSF.

These and other features and advantages of the present invention will be more fully understood from the following detailed description of the invention taken together with the accompanying drawings and the claims. It is noted that the scope of the claims is defined by the recitations therein and not by the specific discussion of features and advantages set forth in the present description.

### BRIEF DESCRIPTION OF THE DRAWINGS

The following detailed description of certain embodiments of this invention can be best understood when read in conjunction with the following drawings, where like structure is indicated with like reference numerals and in which:

FIG. 1 shows a portable external gravity-based device of this invention for regulating CSF drainage amounts from brain, spine, tissue or organs of a patient. As shown in these embodiments, the device of the invention comprises a user interface module (1A) and a fluid-handling module (1B). FIG. 1C) shows an example of how the modules are connected to form the device of the invention. Alternatively, the device of the invention comprises a single module (1D).

FIG. 2 shows the device of the invention comprising an adjustable bracket and the securing screw (2A) for securing the fluid-handling module or a single module of the device of the invention. When the bracket is secured in a horizontal configuration using the bracket-securing screws, the caregiver can attach the fluid-handling module, or in alternative embodiments, the single module of the device of the invention, to a bed rail (2B). When the bracket is secured in a vertical configuration using the bracket-securing screws, the caregiver can attach the fluid-handling module, or in alternative embodiments, the single module of the device of the invention, to an IV pole (2C). FIG. 2C shows the fluid-handling module containing a sensor mechanism, an energy source, a cerebral spinal fluid tubing pathway, a valve mechanism, and a female connector of the electrical connection. FIG. 2D shows a user interface module containing a microprocessor, logic, male connector of the electrical connection, wireless transducer, and a USB port.

FIG. 3A shows the fluid-handling module containing a sensor mechanism, a small flexible compartment for the internal bag. FIG. 3B shows the fluid-handling module containing the sensor mechanism and the CSF tubing. FIG. 3C shows the device of the invention containing Luer locks or other medical connectors, an external drain bag, and a transducer attached to the catheter. FIG. 3D shows the device of the invention connected to the patient and a drain bag, with a transducer attached to the catheter exiting from the ventricle before the inlet opening of the device of the invention. FIG. 3E shows that the device can be “flushed” with the saline solution using an external syringe.

Skilled artisans will appreciate that elements in the figures are illustrated for simplicity and clarity and have not necessarily been drawn to scale. For example, the dimensions of some of the elements in the figures may be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Before describing the present invention in detail, a number of terms will be defined. As used herein, the singular forms “a”, “an”, and “the” include plural referents unless the context clearly dictates otherwise.

As used herein the term “parameter” can mean a string of text, a digital signal, a wave form, a data rate, or a bit rate containing information on flow rate limit, an amount drained last hour, an amount drained since midnight, an amount drained yesterday, and signals received by the logic. The term parameter can further mean other things related to continuous gravity-based CSF drainage.

As used herein, the abbreviation “CSF” means cerebral spinal fluid; the abbreviation “NPH” means Normal Pressure Hydrocephalus; and the abbreviation “EMR” means electronic medical record.

The invention generally provides portable external gravity-based devices for regulating CSF drainage amounts from brain, spine, tissue or organs of a patient (the “device”). External means that the device is located outside of the patient's body. Portable means that a caregiver can move the device manually. Gravity-based means that the device regulates cerebral spinal fluid drainage based on gravity outflow from brain, spine, tissue or organs of a patient without the use of a mechanical, electric, or other type of a pump.

Devices as set forth herein avoid reinfusion of the drained CSF into the brain or spine, which can introduce infection or raise intracranial pressure. Furthermore, the device as set forth herein does not discriminate between CSF drained from the ventricular system of the brain or the intrathecal space of the spinal canal.

The device as set forth herein can be used where CSF drainage is required but not in situations of suspected elevated intracranial pressure. For example, devices as set forth herein are designed to be used in post-neurosurgical or ENT procedures when CSF is encountered and where it is desired to seal the dura mater (the sac which contains the brain, the spinal cord and the CSF) at the surgical site via re-routing the CSF from an alternate pathway in the form of an external ventricular drain or the lumbar drain. Such neurosurgical or ENT procedures include, in non-limiting example, skull base surgery, pituitary surgery, traumatic skull base fractures, and sinus surgery (ethmoid, sphenoid and mastoid).

Furthermore, the device as set forth herein is designed to be used in post spinal surgery, where CSF has been encountered and an intrathecal catheter is placed to seal the duratomy defect.

Yet further, devices as set forth herein are useful for determining whether continuous CSF drainage for 3 days results in clinical improvement in patients with suspected NPH. In cases where external drainage reveals success, an internal shunt can be placed.

However, it will be recognized that using devices as set forth herein are contraindicated in cases where elevated intracranial pressure is suspected. Devices as set forth herein can accommodate measurement of intracranial pressure by having a transducer attached to the catheter exiting the ventricle before the inlet opening. The device is not meant to treat elevated intra-cranial pressure via a liberal and pressure differential fashion, which results in a proportional drainage of CSF without the need to regulate the volume. Examples of situations where the device as set forth herein should not be used are post head trauma, intraventricular hemorrhage, or existence of midline shift in the one hemisphere more than 5 mm to the opposite side.

In preferred embodiments of the invention, the device as set forth herein does not require an electrical outlet, avoiding any chance of electrical injury to the patient.

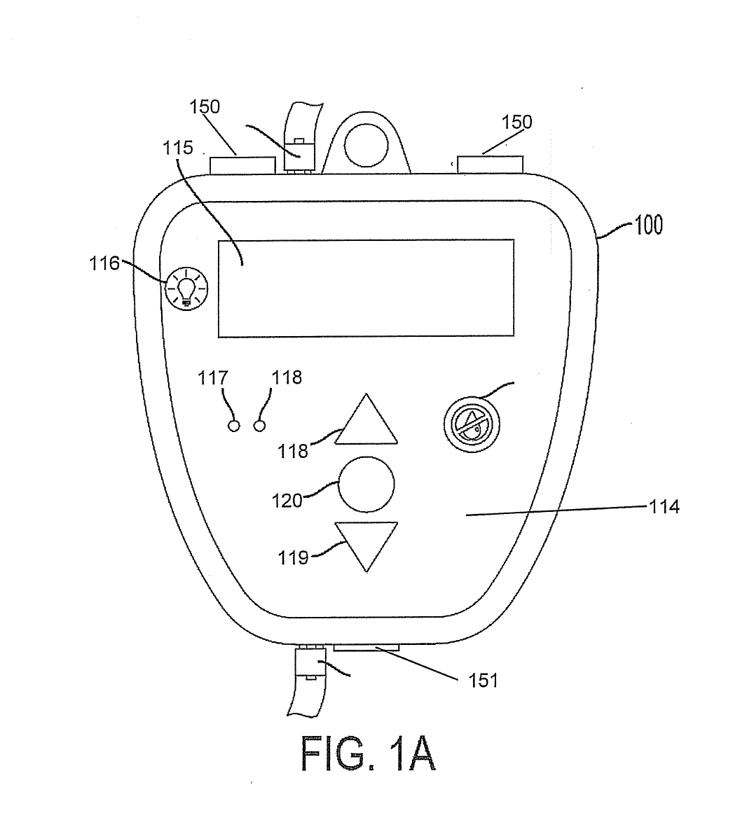

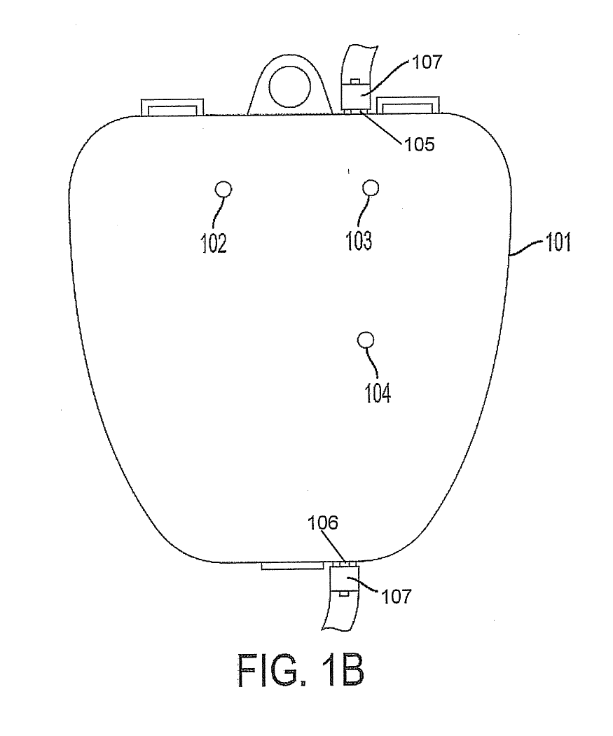

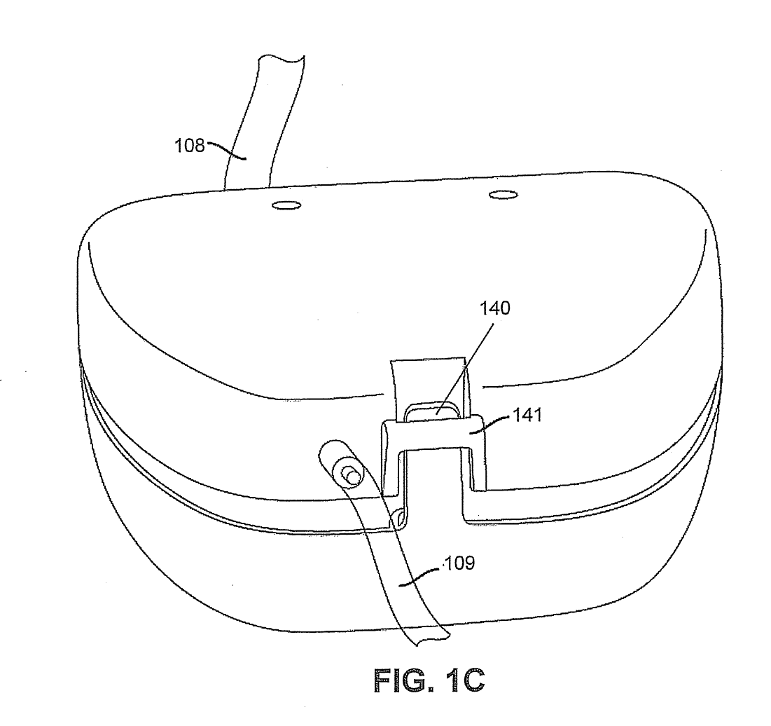

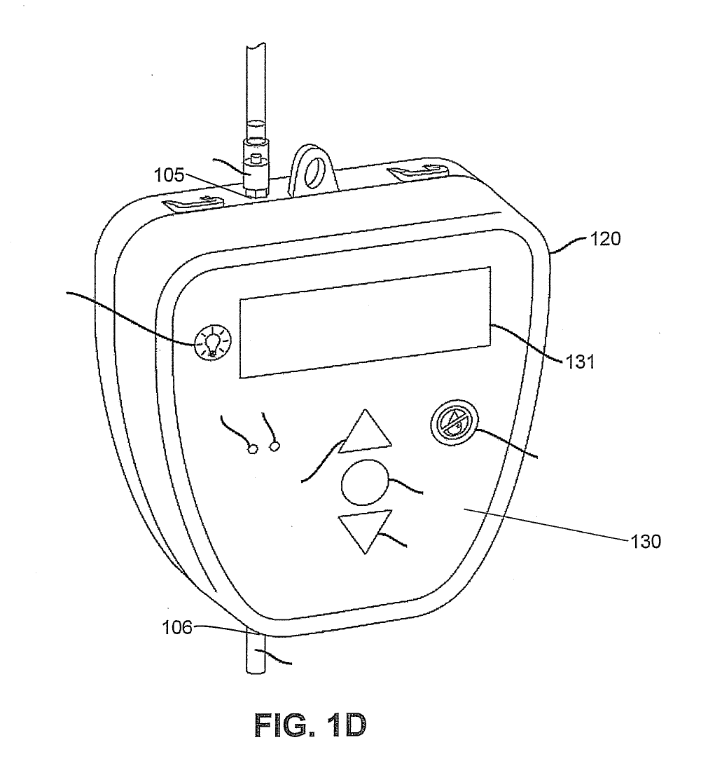

In one embodiment of the invention, the device comprises a user interface module (FIG. 1 A) and a fluid-handling module (FIG. 1 B). The modules are physically connected to form the device through a “tong” and “lock” mechanism. The tong and lock mechanism of a physical connection comprises a first tong and lock mechanism 150, located on the inlet side of the device and a second tong and lock mechanism 151, located on the outlet side of the device (FIG. 1A). In some embodiments, the tong 160 of the first tong and lock mechanism is located on the user interface module and the lock 161 of the first tong and lock mechanism is located on the fluid-handling module (FIG. 1C) and the tong 140 of the second tong and lock mechanism is located on the user interface module and the lock 141 of the second tong and lock mechanism is located on the fluid-handling module (FIG. 1C). In alternative embodiments, the tong of the first and of the second tong and lock mechanism can be located on the fluid-handling module and the lock of the first and in the second tong and lock mechanism can be located on the user interface module. In some embodiment, the tong and the lock of the first and of the second tong and lock mechanism are made of plastic, particularly thermoplastic. In other embodiments, the tong and the lock of the first and of the second tong and lock mechanism are made of other materials including, but not limited to, metal.

In alternative embodiment of the invention, the device comprises a single module (FIG. 1D), comprising logic, input interface, a sensor mechanism, a communication system to communicate a signal from the sensor mechanism, an energy source, a cerebral spinal fluid tubing pathway, and a valve mechanism.

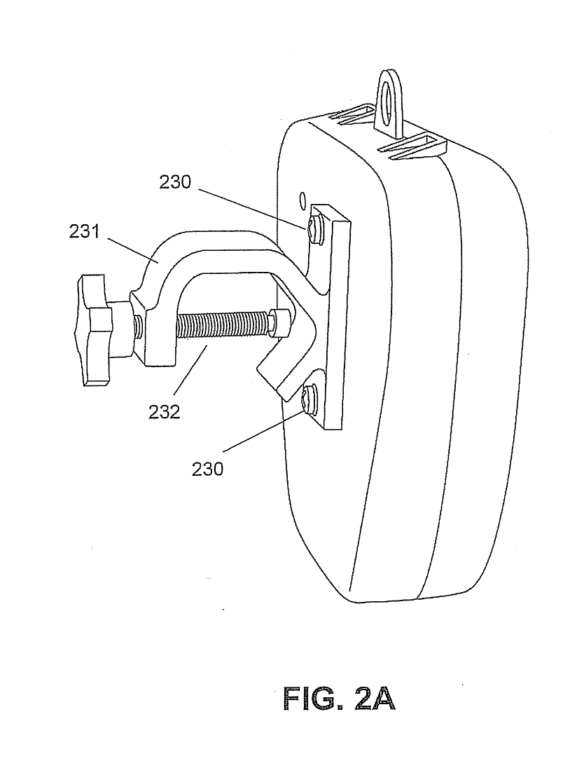

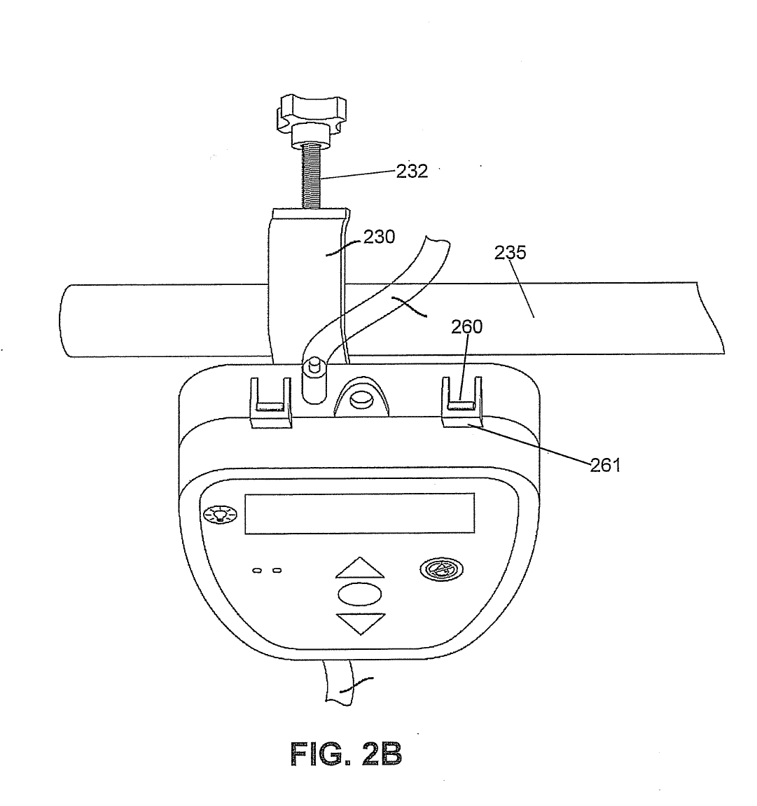

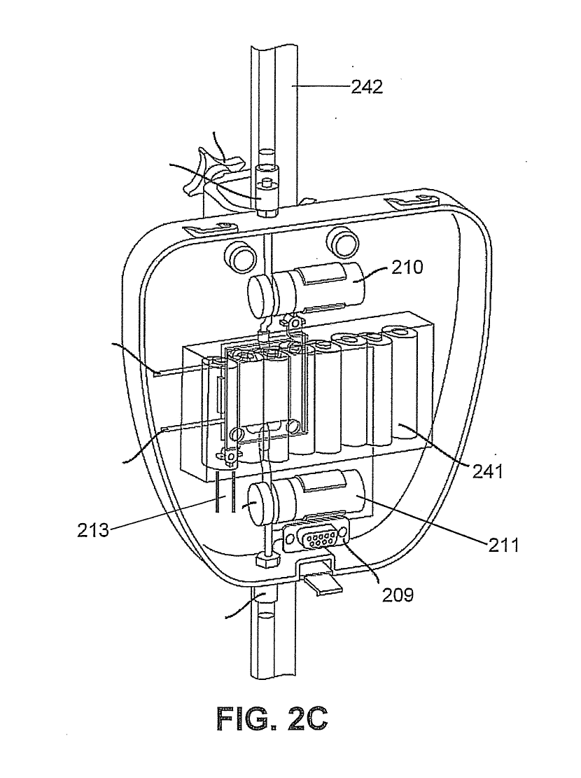

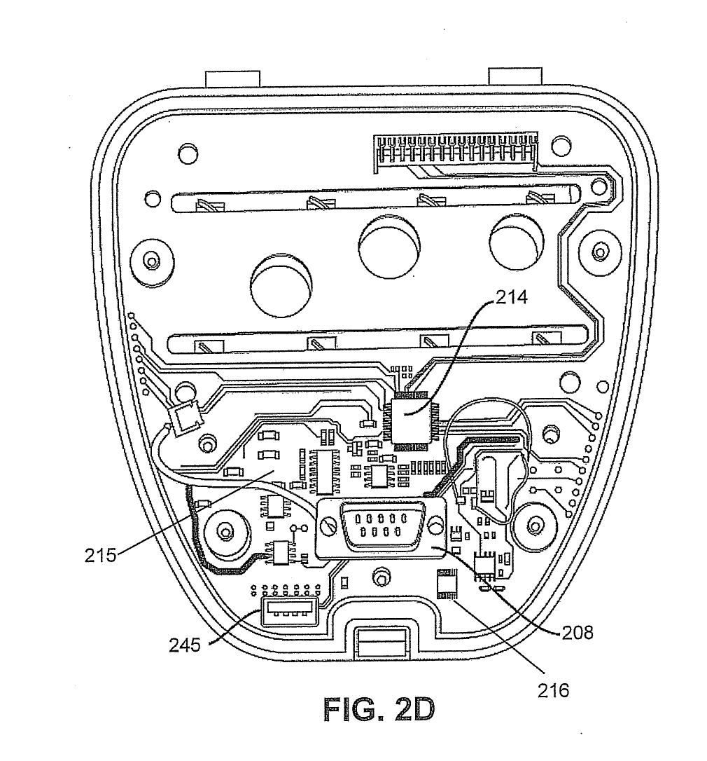

In advantageous embodiments of the invention, the fluid-handling module of the device, or in alternative embodiments, the single module of the device, is sterile and is designed to be discarded after each patient use, inter alia to minimize contamination. The housing of the fluid-handling module, or in alternative embodiments, the housing of the single module of the device, can advantageously comprise an adjustable bracket 231, the fluid-handling module- or single module-securing screw 232 and two bracket-securing screws 230 (FIG. 2A). The adjustable bracket 231, the fluid-handling module- or single module-securing screw 232 and two bracket-securing screws 230 can be used to secure the fluid-handling module, or in alternative embodiments, the single module of the device to a support member. The support member can be, but not limited to, a horizontally-arranged support member, for example, a bed rail or a vertically-arranged support member, for example, an IV pole. When the bracket is secured in a horizontal configuration using the bracket-securing screws 230 (FIG. 2A) and screw openings 102 and 103 (FIG. 1B), the caregiver can attach the fluid-handling module, or in alternative embodiments, the single module of the device, to a bed rail 235 (FIG. 2B) or other horizontally-arranged support member. When the bracket is secured in a vertical configuration using the bracket-securing screws 230 (FIG. 2A) and screw openings 103 and 104 (FIG. 1B), the caregiver can attach the fluid-handling module, or in alternative embodiments, the single module of the device, to an IV pole 242 (FIG. 2C) or other vertically-arranged support member.

In other embodiments of the invention, the fluid-handling module of the device, or in alternative embodiments, the single module of the device, can contain an energy source 241 used for powering the fluid-handling module and the user interface module of the device or the single module of the device (FIG. 2C). The energy source 241 can be a battery, particularly a rechargeable battery, or a capacitor, or any component capable of storing a charge appropriate for powering the device. In preferred embodiments, the battery is an alkaline battery, a lithium battery, or a NiMH battery.

In some embodiments of the invention, the device containing the fluid-handling module and the user interface module as set forth herein comprises an “on/off” switch for controlling the activity state of the device. In other embodiments, the device does not have an on/off switch and the fluid-handling module and the user interface module are operatively connected through an electrical connection, in non-limiting example between a male connector 208 on the user interface module (FIG. 2D) and a female connector 209 on the fluid-handling module (FIG. 2C). In these embodiments, the device is expected to automatically power on when the user interface module and the fluid-handling module are connected and is expected to automatically power off when the user interface module and the fluid-handling module are disconnected.

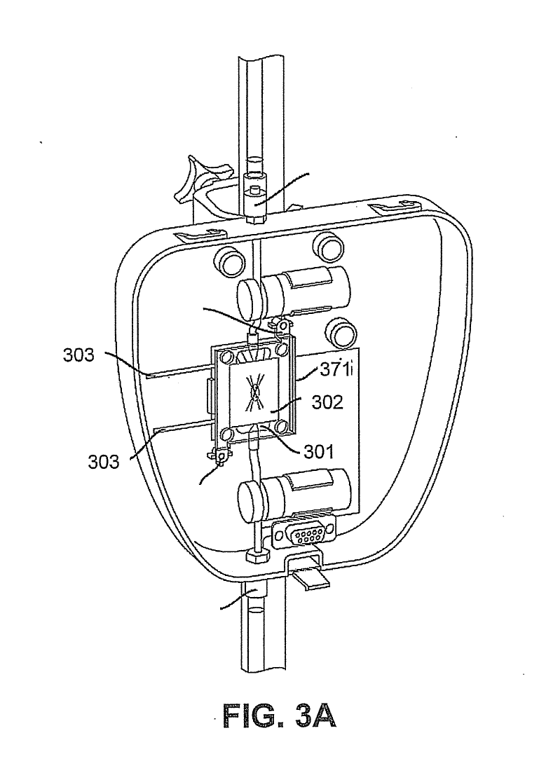

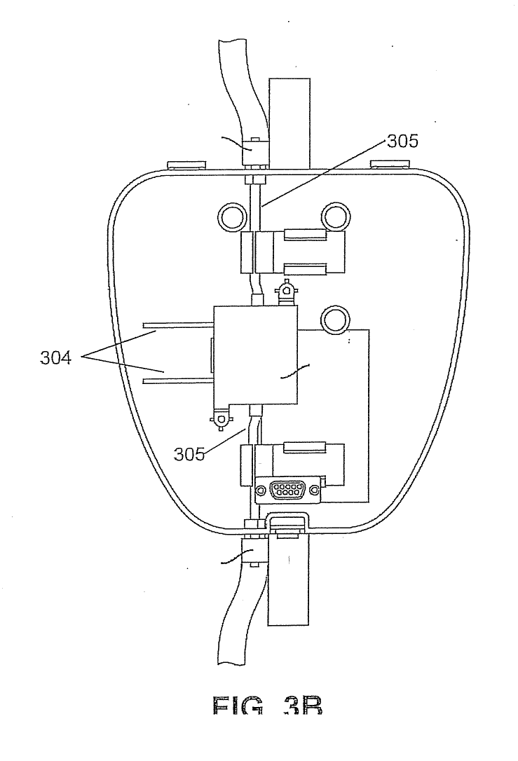

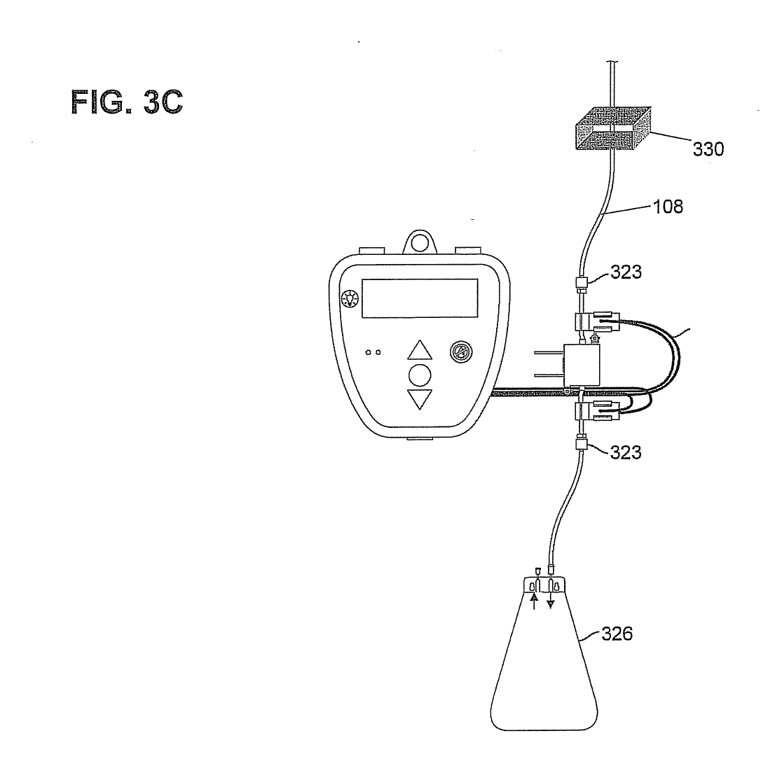

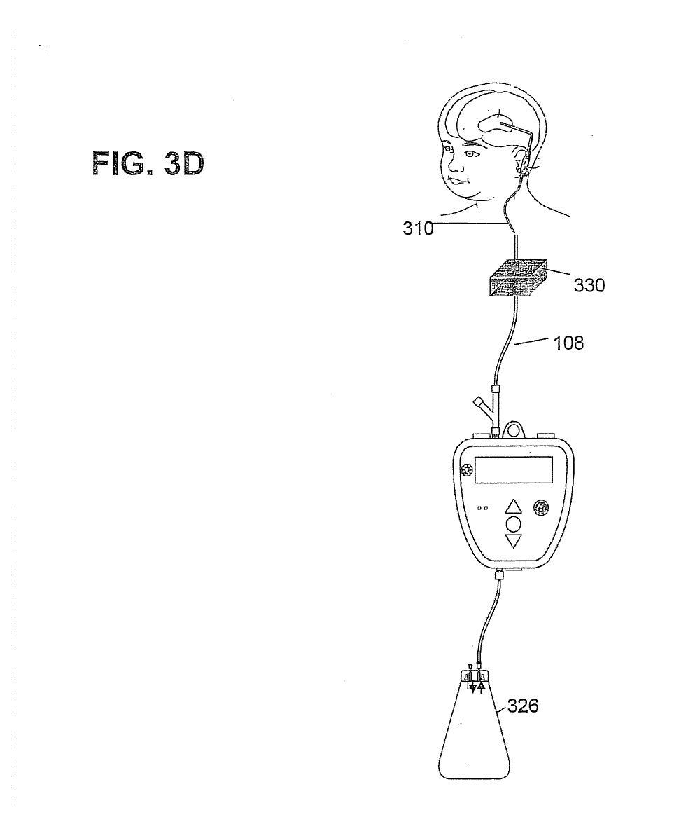

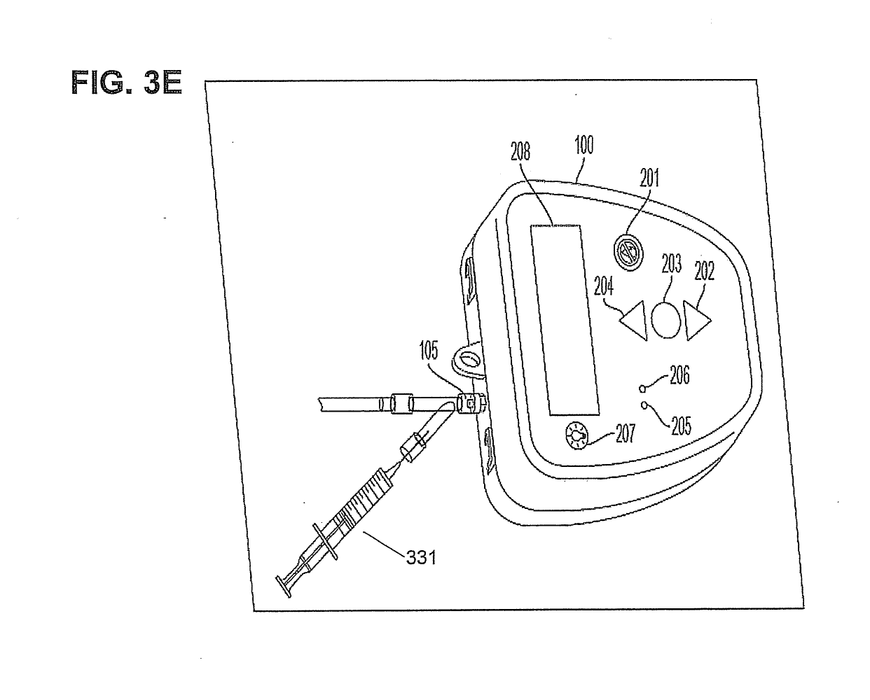

In additional embodiments of the invention, the housing 101 of the fluid-handling module of the device (FIG. 1B), or in alternative embodiments, the housing of the single module of the device 120 (FIG. 1D), is made of plastic, particularly thermoplastic, and contains an inlet opening 105 for coupling to a shunt line and an outlet opening 106 for coupling to a drain line; in certain embodiments, both the inlet and outlet opening are located at the same or adjacent positions on the opposite up and down sides in the housing (FIGS. 1B and 1D). Preferably, coupling of the fluid-handling module to the shunt line 108 and the drain line 109 (FIG. 1C) is via Luer locks 323 (FIG. 3C) or other medical connectors at any point above the drain bag as long as the fluid-handling module is positioned at least 6 inches below the patient's hip, whether the person is sitting, laying, or standing to ensure proper outflow through the module. The device is most advantageously positioned generally upright for generally vertical downward flow of cerebral spinal fluid therethrough.

In further embodiments of the invention, CSF movement through the fluid-handling module of the device, or in alternative embodiments, the single module of the device, is facilitated through a pinch solenoid-operated valve mechanisms 210 and 211 (FIG. 2C) that fills and drains periodically a small flexible compartment or an internal bag 301 (FIG. 3A). The small flexible compartment or the internal bag holds a user-determined amount of CSF and enables the device to empty the user-determined amount of CSF per hour. The valve mechanism of the device comprises a first, or proximal, valve 210 that permits CSF to move from a shunt line 108 (FIG. 1C) into the flexible compartment or the internal bag 301 (FIG. 3A) and a second, or distal, valve 211 that permits CSF to move from the flexible compartment or the internal bag 301 (FIG. 3A) into an external drain bag 326 (FIG. 3D).

In one particular embodiment of the invention, the device limits the flow of cerebrospinal fluid to the external drain bag 326 (FIG. 3D) by periodically filling and emptying the flexible compartment or the internal bag 301 (FIG. 3A) inside the device. Varying rates of drainage can be achieved by varying the timing of the filling/emptying cycle.

In further embodiments of the invention, the internal fluid chamber is drained into the external drain bag 326 (FIG. 3D) by closing the proximal valve 210 and opening the distal valve 211 (FIG. 2C). This mechanism ensures that only 1-2 cc of CSF is exited at any single time point, and while small flexible compartment or the internal bag is being drained no additional CSF is permitted to exit the patient. The amount of CSF drained can be set to no more than 20 cc per hour.

In certain embodiments, external drain bag 326 (FIG. 3D) of a regulated gravity-based cerebral spinal fluid drainage device as set forth herein can be changed as many times as necessary without the need for changing any other components; in such embodiments, the device is configured to have a “pause” mode so CSF is not being drained from the patient or the device or both while changing the external drain bag 326 (FIG. 3D).

In further embodiments, the fluid-handling module of the device, or in alternative embodiments, the single module of the device, contains a sensor mechanism, wherein the sensor mechanism further comprises a first sensor, or a clog sensor, 302 (FIG. 3A) and a second sensor 213 (FIG. 2C), wherein the first sensor 302 is located on the flexible compartment, or the internal bag 301 (FIG. 3A), wherein the first sensor comprise a pair of electrodes 303 used to sense a change in the amount of fluid in the internal bag, and the second sensor is located on the battery (FIG. 2C). The first sensor 302 of the sensor mechanism detects if the cerebral spinal fluid tubing pathway 305 is clogged and sends a signal to the user interface module (FIG. 1A) if the change in the amount of fluid in the internal bag is lower than the user predetermined value, wherein the alarm is sounded to alert the user. The second sensor 213 of the sensor mechanism detects a low battery condition and sends a signal to the user interface module (FIG. 1A), wherein the alarm is sounded to alert the user.

In some embodiments, the user interface module of the device (FIG. 1A) is a non-sterile and a reusable part of the device and contains logic, input interface 114 (FIG. 1A), and a communication system to communicate the signal from the sensor mechanism of the fluid-handling module. The housing 100 of the user interface module can be made of plastic, and in particular thermoplastic. In alternative embodiment, the single module of the device contains logic, input interface 130 (FIG. 1D), and a communication system to communicate the signal from the sensor mechanism to the user.

The communication system of the user interface module of the device, or in alternative embodiments, of the single module of the device, generally contains a display system that communicates a signal to a user. The display system of the user interface module of the device, or in alternative embodiments, of the single module of the device, comprises a display screen 115 (FIG. 1A) that displays parameter information to the user. Parameter information can generally include but is not limited to current flow rate limit, an amount drained last hour, an amount drained since midnight, and an amount drained yesterday. The communication system of the user interface module of the device, or in alternative embodiments, of the single module of the device, alerts the caregiver that the system is functioning normally by an observable signal, for example a green light-emitting diode 117 (FIG. 1A) that flashes periodically on the communication system. The communication system of the user interface module of the device, or in alternative embodiments, of the single module of the device, also alerts the caregiver that the logic has received a signal from the sensor mechanism indicating, for example, a clogged cerebral spinal fluid tubing pathway signal from the sensor, or that the current flow rate limit parameter is outside of the user-predetermined flow rate range, or that a low battery condition exists, preferably by a preferably different detectable signal, for example a red light-emitting diode 118 (FIG. 1A) that flashes continuously, or an audible signal such as an alarm, or both.

The input interface of the device allows a user to increase or decrease the cerebral spinal fluid drainage limit and to make menu selections, preferably from a menu range restricted to permissible performance ranges. Furthermore, the input interface of the device is used to set a fill/drain timing schedule to increase or decrease the amount of cerebral spinal fluid drained, a user-predetermined flow rate range or a time of day. The input interface of the device can comprise dedicated inputs, for example buttons, for changing menu parameters. In non-limiting examples, these can include an “up” button 118 to increase the cerebral spinal fluid drainage limit, a “down” button 119 to decrease the cerebral spinal fluid drainage limit, and a “select” button 120 to make menu selections (FIG. 1A).

The logic of the user interface module of the device, or in alternative embodiments, of the single module of the device, is generally configured to receive, display or process a signal from the sensor mechanism of the fluid-handling module or the input interface, or to cause an action to be taken.

In some embodiments, the logic comprises a microprocessor 214 (FIG. 2D), wherein the microprocessor further comprises memory, wherein the memory includes instructions that, when executed by the microprocessor causes the microprocessor to receive, display, store or process signals from the sensor mechanism or the input interface, or to cause an action to be taken.

In some embodiments, the logic comprises random access memory that includes instructions that, when executed causes the logic to receive, display, store or process signals from the sensor mechanism or the input interface, or to cause an action to be taken.

In some embodiments, the logic is configured to regulate the amount of cerebral spinal fluid drained to no more than 20 cubic centimeters per hour to receive signals from the sensor mechanism of the fluid-handling module comprising a current flow rate limit parameter, a clogged cerebral spinal fluid tubing pathway signal or a low battery condition signal, and to compare the current flow rate limit parameter received in signals from the sensor mechanism with the user-predetermined flow rate range.

In some embodiments, the device as set forth herein contains certain safety features. For example, the device advantageously contains an alarm that is activated and is sounded and a LED light 118 (FIG. 1A) that becomes activated when the current flow rate limit parameter received in the signal from the sensor mechanism is outside the user-predetermined flow rate range, when a clogged cerebral spinal fluid tubing pathway signal is received from the sensor mechanism, or when a low battery condition signal is received from the sensor mechanism. The clog sensor can be a capacitance sensor, a weight sensor, a flow sensor, a strain gauge sensor, a potentiometric sensor, an optical sensor, or a magnetic sensor.

In some embodiments, the clog sensor 302 can consist of two conductive plates that are placed with the small flexible compartment or an internal bag 301 between them. A box-shaped holder 371 is manufactured from, but is not limited to, thermoplastic, and has an integral lid attached via a living hinge. In some embodiments, the two conductive plates are affixed, one to the inside rear surface of the box, and one to the underside of the lid such that when the fluid bag is inserted into the box and the lid closed, the bag is sandwiched between the conductive plates. The dimensions are optimized so that when the flexible fluid bag is filled to the maximum it touches both plates. The device measures the capacitance between the two plates by energizing one of the electrodes with a step voltage and monitoring the time it takes for the voltage on the second plate to reach a predetermined level. The elapsed time is directly proportional to the amount of fluid within the bag since the dielectric constant of the fluid is much higher than that of air. The time is measured by starting a timer at the same moment that the first plate is energized and using a sensitive comparator to stop the timer when the voltage on the second plate reaches the predetermined level.

In some embodiments, the user interface module of the device, or in alternative embodiments, the single module of the device contains electronics 215 (FIG. 2D) to control the logic, the communication system, the input interface, and the fluid-handling module.

In some embodiments, the device can accommodate measurement of intracranial pressure, for example, by further comprising a transducer 330 attached to the catheter 310 exiting from the ventricle before the inlet opening of the fluid-handling module, or in alternative embodiments, of the single module of the device (FIG. 3 D).

In some embodiments, the transducer can communicate pressure information to the user interface module of the device, or in alternative embodiments, to the single module of the device, via logic, wherein the pressure information is displayed in a manner that the user can easily access it, for example on the display screen directly or in a paper record such as a print-out of the information displayed on the display screen. The user interface module of the device, or in alternative embodiments, the single module of the device, can also notify the user of the pressure information via a wireless transmitter 216 (FIG. 2D).

In some embodiments, logic of the user interface module, or in alternative embodiments, of the single module of the device is configured to be capable of setting, activating, and sounding an alarm to alert a user when pressure rises above a user-predetermined pressure range.

In some embodiments, the user interface module of the device, or in alternative embodiment, of the single module of the device contains a USB port 245 or a wireless transmitter 216 (FIG. 2D). The USB port or a wireless transmitter can be used, for example, for connecting to an external computer system for uploading parameter information, which can generally include but is not limited to current flow rate limit, CSF amounts drained in a particular time period (for example, within the last hour or from a particular set time, such as the amount drained since midnight or on a given day or prior day), and signals received by the logic, into an EMR.

Since the CSF drainage device provided by the invention as set forth herein is regulated, gravity-based device, use of this device avoids reinfusion of the drained CSF into the brain or spine and prevents introduction of infectious microorganisms or an increase of intracranial or intra spinal pressure.

It should be recognized that these inventions also provides embodiments containing varying combinations or all of these expressly-recited features.

The Examples that follow are illustrative of specific embodiments of the invention, and various uses thereof. They set forth for explanatory purposes only, and are not to be taken as limiting the invention.

### EXAMPLES

### Example 1

### External Regulation of Amounts of Gravity-Based Cerebral Spinal Fluid Drainage from Brain, Spine, Tissue or Organs of a Patient Using the Device Containing a Fluid-Handling Module and a User Interface Module

The Example sets forth a method that a caregiver implements for a patient requiring CSF drainage from brain, spine, tissue or organs, for externally regulating the amount of gravity-based CSF drainage.

First, the caregiver obtains a sterile fluid-handling module of a device, as described herein, and preferably utilizing an adjustable bracket 231, the fluid-handling module-securing screw 232, and two bracket-securing screws 230 (FIG. 2A) attach the fluid-handling module of a device to a support member that can be, but not limited to, a horizontally-arranged support member, for example, a bed rail or a vertically-arranged support member, for example, an IV pole. When the bracket is secured in a horizontal configuration using the bracket-securing screws 230 (FIG. 2A) and screw openings 102 and 103 (FIG. 1B), the caregiver can attach the fluid-handling module to a bed rail 235 (FIG. 2B). When the bracket is secured in a vertical configuration using the bracket-securing screws 230 (FIG. 2A) and screw openings 103 and 104 (FIG. 1B), the caregiver can attach the fluid-handling module to an IV pole 242 (FIG. 2C).

The caregiver then physically connects the user interface module to the fluid-handling module through a tong and lock mechanism of the physical connection; and operatively connect the user interface module to the fluid-handling module through an electrical connection established between the two modules; for example male connector 208 on the user interface module (FIG. 2D) and a female connector 209 on the fluid-handling module (FIG. 2C). The user interface module is powered once the fluid-handling module is connected to the user interface module, and the device instructs the user to prime the system or flush the cerebral spinal fluid tubing pathway inside the fluid-handling module with 5 cc saline solution using an external syringe 331 connected to the inlet opening 105 (FIG. 3E) the first time the device is used in a patient; other instructions, inter alia, to set the time and the amount of hourly drainage desired, are communicated through instructions displayed on the display screen.

The caregiver then couples the device to a shunt line 108 (FIGS. 3C,D) of an external ventricular drain (EVD) exiting from the skull or a lumbar drain (LD) exiting from the lumbar spine through the inlet opening 105 of the fluid-handling module of the device, and would couple the drain line 109 to the outlet opening 106 of the fluid-handling module (FIGS. 1B and 1D). Coupling of the fluid-handling module to the shunt line and the drain line is achieved preferably using Luer locks 323 (FIG. 3C) or other medical connectors at any point above the drain bag, provided that the fluid-handling module is positioned at least six inches below the patient's hip to ensure proper outflow through the module. A position six inches below a patient's creates a pressure difference between the CSF-containing structure in the patient (skull or spinal column) and the drainage bag resulting in exit of CSF from the body in a regulated, gravity-based fashion. The fluid-handling module is positioned in a generally upright position for a generally vertical downward flow of CSF therethrough.

As required or desired, the caregiver uses user interface inputs, for example an “up” button, a “down” button, and a “select” button of the input interface (FIG. 1A) to set a fill/drain timing schedule to increase or decrease the amount of CSF drainage required, as well as a volume of CSF drained or a time-of-day setting. The caregiver can also set a range for the amount of CSF drainage required.

When measurement of intracranial pressure is required, a caregiver can use a transducer 330 attached to, in one embodiment, the catheter 310 exiting from the ventricle or, in alternative embodiments, the shunt line 108, and positioned before the inlet opening of the fluid-handling module (FIG. 3D). The transducer communicates the pressure information to the user interface module via logic, wherein the pressure information is displayed preferably on the display screen.

Once the device is powered on, valves 210 and 211 (FIG. 2C), which are, but not limited to pinch solenoid-operated valves, controlled by the logic open and close opposite to each other within a set interval time allowing a limited amount of the CSF to fill the small flexible compartment or an internal bag (1 cc or 2 cc) before shutting off. For example when the CSF is exiting out of the patient's body, the proximal valve 210 is open and the distal valve 211 closed. (FIG. 2C).

A caregiver preferably monitors operation of the device and obtains feed-back information therefrom, such as the amount of CSF exited last hour, the amount of CSF exited since the connection of the device to the patient, the amount of CSF drained in the past hour, and signals received by the logic from the device itself, for example from the display of the communication system of the device.

Furthermore, the caregiver is able to sample CSF from the regulated gravity-based cerebral spinal fluid drainage device for inter alia microbiology and chemistry purposes from the exiting tubing system.

### Example 2

### External Regulation of Amounts of Gravity-Based Cerebral Spinal Fluid Drainage from Brain, Spine, Tissue or Organs of a Patient Using the Device Containing a Single Module

This Example further describes how a caregiver can implement the methods of the invention for patients requiring CSF drainage from brain, spine, tissue or organs for externally regulating the amount of gravity-based CSF drainage.

First, the caregiver obtains a sterile device as described herein, and preferably utilizing an adjustable bracket 231, the single module-securing screw 232, and two bracket-securing screws 230 (FIG. 2A) attach the single module of a device to a support member that can be, but not limited to, a horizontally-arranged support member, for example, a bed rail or a vertically-arranged support member, for example, an IV pole. When the bracket is secured in a horizontal configuration using the bracket-securing screws 230 (FIG. 2A) and screw openings 102 and 103 (FIG. 1B), the caregiver can attach the single module of a device to a bed rail 235 (FIG. 2B). When the bracket is secured in a vertical configuration using the bracket-securing screws 230 (FIG. 2A) and screw openings 103 and 104 (FIG. 1B), the caregiver can attach the single module of a device to an IV pole 242 (FIG. 2C).

Once the device is properly positioned and attached to a support member, the caregiver powers on the device using the on/off switch. Once the device is powered on, the device instructs the user to prime the system or flush the cerebral spinal fluid tubing pathway inside the fluid-handling module with 5 cc saline solution using an external syringe 331 connected to the inlet opening 105 (FIG. 3E) the first time the device is used in a patient; similar instructions, inter alia, to set the time and the amount of hourly drainage desired, are communicated through instructions displayed on the display screen.

The caregiver then couples the device to a shunt line 108 (FIG. 3C) of an external ventricular drain (EVD) exiting from the skull or a lumbar drain (LD) exiting from the lumbar spine through the inlet opening 105 of the fluid-handling module of the device, and couples the drain line 109 to the outlet opening 106 of the fluid-handling module (FIGS. 1B and 1D). Coupling of the fluid-handling module to the shunt line and the drain line is achieved preferably using Luer locks 323 (FIG. 3C) or other medical connectors at any point above the drain bag, provided that the fluid-handling module is positioned at least six inches below the patient's hip to ensure proper outflow through the module by gravity. A position six inches below a patient's creates a pressure difference between the CSF-containing structure in the patient (skull or spinal column) and the drainage bag resulting in exit of CSF from the body in a regulated, gravity-based fashion. The fluid-handling module is positioned in a generally upright position for a generally vertical downward flow of CSF therethrough.

As required or desired, the caregiver uses user interface inputs, for example an “up” button, a “down” button, and a “select” button of the input interface (FIG. 1A) to set a fill/drain timing schedule to increase or decrease the amount of CSF drainage required, as well as a volume of CSF drained or a time-of-day setting. The caregiver can also set a range for the amount of CSF drainage required.

When measurement of intracranial pressure is required, a caregiver can utilize a transducer 330 attached to the catheter 310 exiting from the ventricle and positioned before the inlet opening of the fluid-handling module (FIG. 3D). The transducer communicates pressure information to the user interface module via logic, wherein the pressure information is displayed preferably on the display screen.

Once the device is powered on, valves 210 and 211 (FIG. 2C), which are, but not limited to pinch solenoid-operated valves, controlled by the logic open and close opposite to each other within a set time interval allowing a limited amount of the CSF to fill the small flexible compartment or an internal bag (1 cc or 2 cc) before shutting off. For example when the CSF is exiting out of the patient's body, the proximal valve 210 is open and the distal valve 211 closed. (FIG. 2C).

A caregiver preferably monitors operation of the device and obtains feed-back information from the display of the communication system of the device, such as the amount of CSF exited last hour, the amount of CSF exited since the connection of the device to the patient, the amount of CSF drained in the past hour, and signals received by the logic from the device itself, for example.

Furthermore, the caregiver is able to sample CSF from the regulated gravity-based cerebral spinal fluid drainage device for microbiology and chemistry purposes from the exiting tubing system.

### Example 3

### Reducing Secondary Brain Injury of a Patient

Following the steps outlined in Example 1 or 2, a caregiver can establish external regulation of amounts of gravity-based cerebral spinal fluid drainage from brain, spine, tissue or organs of a patient. The regulated gravity-based cerebral spinal fluid drainage device does not discriminate between the CSF drained from the ventricular system of the brain or the intrathecal space of the spinal canal.

Since the regulated gravity-based cerebral spinal fluid drainage device of the instant invention is gravity-based, the use of a device of the invention avoids reinfusion of the drained CSF into the brain or spine and prevents introduction of infection or increase of the intracranial or intra spinal pressure. This reduces potential secondary brain injury of a patient since the system is gravity based it does not require active drainage, which can result in unwanted side effects, such as bleeding in the subdural space or suctioning of the nerve root into the system.

### Example 4

### External Regulation of a Continuous Gravity-Based Cerebral Spinal Fluid Drainage from a Brain, a Spine, a Tissue or an Organ of a Patient with Suspected NPH

Following the steps outlined in Example 1 or 2, a caregiver can establish external regulation of the amount of gravity-based cerebral spinal fluid drainage from brain, spine, tissue or organs of a patient.

A caregiver can determine whether continuous drainage of CSF for 3 days results in clinical improvement in patients with suspected NPH. In case the external drainage reveals success, then an internal shunt can be placed. A lumbar drain (LD) catheter 310 (FIG. 3C) is first inserted and then connected to the device using Luer locks 323 (FIG. 3C) or other medical connectors. Since this device allows patients to walk, continuous drainage of CSF allows the clinician/physical therapist to determine if the patient's condition is drainage sensitive and if so an internal ventriculo peritoneal or Lumbo-peritoneal shunt is inserted.

### Example 5

### Use of External Gravity-Based Cerebral CFS Drainage Device in Cases of CSF Leak in Neurosurgical, ENT, and Orthospine Surgeries

In patients who have had surgical interventions crossing the dural lining, there will be chances of post-operative CSF leakage. The clinical situations for such a condition include neurosurgical posterior fossa surgeries, for example, tumor resections, vascular procedures, chiari decompression with dural patch reconstruction, ENT procedures with the endoscopic packing of the ethmoidal and sphenoid sinus for the conditions of the CSF leak from incompetent cribriform plate or mastoidectomy and spinal procedures by neurosurgeons or orthopedics surgeons, for example lumbar microdiscectomies, lumbar fusion, intradural tumor resection, in which the hydrostatic pressure of the CSF column can result in leakage of the CSF from the duratomy site and the skin.

In above situations, 3 to 7 days CSF drainage in a regulated fashion of no more than 20 cc/hour from a lumbar drain or external ventricular drain is ideal in order to allow for the proper healing of the duratomy defect.

### Example 6

### Uploading Parameter Information into an Electronic Medical Record (EMR)

Following the steps outlined in Example 1 or 2, a caregiver can establish external regulation of the amount of gravity-based cerebral spinal fluid drainage from brain, spine, tissue or organs of a patient.

A caregiver obtains feedback information on the amount of CSF exited from the patient (for example, over the last hour, since the connection of the device to the patient, as well as signals received by the logic from the display of the communication system of the device). In particular illustrations of the method, signals received by the logic are, for example, a clogged cerebral spinal fluid tubing pathway signal from the sensor mechanism, a signal from the fluid-handling module that the current flow rate limit parameter is outside of the user-predetermined flow rate range, or a low battery condition signal from the sensor mechanism.

A caregiver can obtain intracranial pressure information from the display of the communication system when the device is configured to have a transducer 330 (FIG. 3C) attached to the catheter exiting from the ventricle before the inlet opening of the fluid-handling module, wherein the transducer communicates the intracranial pressure information to the user interface module via logic.

When an external regulated gravity-based cerebral spinal fluid drainage device of this invention contains a USB port 245 or a wireless transmitter 216 (FIG. 2D), the caregiver can establish a USB or a wireless connection between the device and an external computer system.

With these capabilities, the caregiver can upload into an EMR parameter information as described above including signals received by the logic.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. A device for regulating cerebral spinal fluid drainage amounts from brain, spine, tissue or organs of a patient comprising:
a user interface module comprising logic, an input interface, a communication system to communicate a signal from a sensor mechanism of a fluid-handling module, a first component of a physical connection, and a first component of an electrical connection, and
the fluid-handling module comprising a sensor mechanism, an energy source, a cerebral spinal fluid tubing pathway, a valve mechanism, a second component of the physical connection and a second component of the electrical connection, wherein the user interface module and the fluid-handling module are operatively connected through the first component and the second component of the electrical connection,
wherein the device is portable external gravity-based device.

2. The device of claim 1, wherein the fluid-handling module contains an energy source for powering itself and the user interface module, wherein the energy source is a battery, is rechargeable or is a capacitor, wherein the battery is an alkaline battery, a lithium battery or a NiMH battery.

3. The device of claim 1, wherein the device has an on/off switch.

4. The device of claim 1, wherein the device is automatically powered on when the user interface module and the fluid-handling module are connected, and wherein the device is automatically powered off when the user interface module and the fluid-handling module are disconnected.

5. The device of claim 1, wherein the fluid-handling module comprises a housing having an inlet opening and an outlet opening, wherein the inlet opening is adapted to be coupled to a shunt line and the outlet opening is adapted to be coupled to a drainage line, wherein the fluid-handling module is adapted to be coupled to the shunt line and the drainage line via Luer locks or other medical connectors, wherein the fluid-handling module is sterile and disposable.

6. The device of claim 1, wherein the valve mechanism is a pinch solenoid-operated valve mechanism that fills and periodically drains a small flexible compartment or an internal bag, further comprising a first valve, wherein the first valve allows drainage of the cerebral spinal fluid from a shunt line into the flexible compartment or the internal bag, and a second valve, wherein the second valve allows drainage of the cerebral spinal fluid from the flexible compartment or the internal bag into an external drain bag.

7. The device of claim 6, wherein the small flexible compartment or the internal bag holds a pre-determined amount of CSF and the device controls the rate the small flexible compartment or the internal bag is filled and drained, wherein the device is capable of emptying the user-determined amount of CSF each hour.

8. The device of claim 1, wherein the sensor mechanism further comprises a first sensor and a second sensor, wherein the first sensor is located on the flexible compartment, or the internal bag, wherein the first sensor comprise a pair of electrodes used to sense a change in the amount of fluid in the flexible compartment or the internal bag, and the second sensor is located on the battery, wherein the first sensor of the sensor mechanism detects when the cerebral spinal fluid tubing pathway is clogged and sends a signal to the user interface module if the change in the amount of fluid in the internal bag is lower than a user predetermined value, wherein the second sensor of the sensor mechanism detects a low battery condition and sends a signal to the user interface module.

9. The device of claim 1, wherein the housing of the fluid-handling module comprises an adjustable bracket, a fluid-handling module-securing screw, and two bracket-securing screws, wherein the bracket in a vertical configuration is capable of attaching to a support member, by adjusting the bracket placement, wherein the bracket in a horizontal configuration is capable of attaching to a support member, by adjusting the bracket placement.

10. The device of claim 1, where the logic is configured to receive, display or process a signal from the sensor mechanism of the fluid-handling module or the input interface, or to cause an action to be taken, to regulate the amount of cerebral spinal fluid drained to no more than 20 cubic centimeters per hour or to compare the current flow rate limit parameter received in the signal from the sensor mechanism of the fluid-handling module with the user-predetermined flow rate range.

11. The device of claim 1, wherein the logic comprises a microprocessor, wherein the microprocessor further comprises memory, wherein the memory includes instructions that, when executed by the microprocessor causes the microprocessor to receive, display, store or process the signal from the sensor mechanism of the fluid-handling module or the input interface, or to cause action to be taken, wherein the signal from the sensor mechanism of the fluid-handling module comprises a current flow rate limit parameter, a clogged cerebral spinal fluid tubing pathway signal or a low battery condition signal.

12. The device of claim 1, wherein the logic comprises random access memory, wherein the random access memory includes instructions that, when executed causes the logic to receive, display, store or process the signal from the sensor mechanism of the fluid-handling module or the input interface, or to cause an action to be taken.

13. The device of claim 1, wherein the input interface is used to set a fill/drain timing parameter to increase or decrease the amount of cerebral spinal fluid drained, a use-predetermined flow rate range or a time of day.

14. The device of claim 1, wherein the logic comprises an alarm, wherein the logic activates and sounds the alarm to alert a user when the current flow rate limit parameter received in the signal from the sensor mechanism of the fluid-handling module is outside of the user-predetermined flow rate range, to alert a user when a clogged cerebral spinal fluid tubing pathway signal is received from a clog sensor in the fluid-handling module, wherein the clog sensor is a capacitance sensor, a weight sensor, a flow sensor, a strain gauge sensor, a potentiometric sensor, an optical sensor or a magnetic sensor, or to alert a user when a low battery condition signal is received from the fluid-handling module.

15. The device of claim 1, wherein the communication system comprises a display system that communicates the signal to a user, wherein the display system further comprises a display screen that displays parameter information to the user, wherein the parameter information displayed to the user includes the current flow rate limit, an amount drained over a time interval.

16. The device of claim 15, wherein the display system further comprises a light-emitting diode that flashes periodically to indicate that the system is functioning normally.

17. The device of claim 15, wherein the display system further comprises a light-emitting diode that flashes continuously to indicate that the logic has received a clogged cerebral spinal fluid tubing pathway signal from the fluid-handling module, or a signal from the fluid-handling module that the current flow rate limit parameter is outside of the user-predetermined flow rate range, or a low battery condition signal from the fluid-handling module.

18. The device of claim 1, wherein the input interface further comprises a member to increase the cerebral spinal fluid drainage limit, a member to decrease the cerebral spinal fluid drainage limit, and a member to make menu selections.

19. The device of claim 1, wherein the user interface module further comprises electronics to control the logic, the communication system, the input interface, and the fluid-handling module.

20. The device of claim 1, wherein the device regulates the amount of gravity-based cerebral spinal fluid drainage from the ventricular system of the brain or the intrathecal space of the spinal canal.

21. The device of claim 1, wherein the device further comprises a pressure transducer attached to the catheter exiting from the ventricle before the inlet opening of the fluid-handling module and is adapted for measuring intracranial pressure by the transducer, wherein the transducer communicates pressure information to the user interface module via logic, wherein the pressure information is displayed on the display screen, wherein the logic sets, activates and sounds the alarm to alert a user when the pressure is above the user-predetermined pressure range.

22. The device of claim 1, wherein the user interface module contains a USB port or a wireless transmitter.

23. A method of externally regulating gravity-based cerebral spinal fluid drainage from brain, spine, tissue or organs of a patient, comprising the steps of:
positioning the fluid-handling module of a device of claim 1 at least six inches below the patient's hip to ensure proper outflow through the module by gravity;
attaching the fluid-handling module of the device to a support member;
connecting the fluid-handling module of the device to the user interface module, and thereafter powering the user interface module;
flushing the cerebral spinal fluid tubing pathway of the device with 5 cc saline solution using an external syringe the first time the device is being used in a patient;
coupling the fluid-handling module of the device to the shunt line through the inlet opening and to the drain line through the outlet opening; and
using the logic of the device to set a fill/drain timing schedule to increase or decrease the amount of cerebral spinal fluid drainage required, a volume of CSF drained, or time of day setting or to control the device by setting the range for the cerebral spinal fluid drainage required,
wherein the method is external gravity-based method.

24. The method of claim 23, wherein the gravity-based cerebral spinal fluid drainage from brain, spine, tissue or organs of a patient is continuous.

25. The method of claim 24, further comprising the step of determining if the continuous drainage of CSF for 3 days results in clinical improvement in patients with suspected Normal Pressure Hydrocephalus (NPH).

26. The method of claim 23, further comprising the step of reducing secondary brain injury of a patient by externally regulating the amount of gravity-based cerebral spinal fluid drainage from brain or spine of a patient to prevent subdural hematoma.

27. A method for improving post-operative outcomes in a patient following a neurosurgical or ENT procedure using a device according to claim 1, wherein the post neurosurgical or the ENT procedure are triggered by the CSF encounter and wherein sealing of a dura mater at a surgical site via re-routing of the CSF from an alternate pathway in the form of an external ventricular drain or the lumbar drain, wherein the dura mater is a sac which contains the brain, spinal cord and CSF, comprising the steps of:
positioning the fluid-handling module of a device of the invention at least six inches below the patient's hip to ensure proper outflow through the module by gravity;
attaching the fluid-handling module of the device to a support member;
connecting the fluid-handling module of the device to the user interface module, and thereafter powering the user interface module;
flushing the cerebral spinal fluid tubing pathway of the device with 5 cc saline solution using an external syringe the first time the device is being used in a patient;
coupling the fluid-handling module of the device disclosed herein to the shunt line through the inlet opening and to the drain line through the outlet opening; and
using the logic of the device to set a fill/drain timing schedule to increase or decrease the amount of cerebral spinal fluid drainage required, a volume of CSF drained, or time of day setting or to control the device by setting the range for the cerebral spinal fluid drainage required,
wherein the method is external gravity-based method.

28. The method of use of claim 27, wherein the post neurosurgical or the ENT procedure is a skull base surgery, a pituitary surgery, a traumatic skull base fractures, a post spinal surgery, a sinus surgery, wherein the sinus surgery is a ethmoid, sphenoid or mastoid.

29. A method for uploading parameter information from a device according to claim 22 into an electronic medical record (EMR) comprising the steps of:
establishing a USB or a wireless connection between the device and an external computer system, wherein the external computer system contains the electronic medical record; and
uploading the parameter information from the device into the electronic medical record of the external computer system.

30. The method of claim 29, wherein the parameter information includes the current flow rate limit, an amount drained over a time interval, wherein the parameter information further includes a signal received by the logic, wherein the signal is a clogged cerebral spinal fluid tubing pathway signal from the fluid-handling module, the signal from the fluid-handling module that the current flow rate limit parameter is outside of the user-predetermined flow rate range or the low battery condition signal from the fluid-handling module.

31. A fluid-handling module comprising a housing, a sensor mechanism, an energy source, a cerebral spinal fluid tubing pathway, a valve mechanism, and a component of an electrical connection, wherein the energy source is a battery, is rechargeable or is a capacitor, wherein the battery is selected from the group comprising an alkaline battery, a lithium battery, and a NiMH battery.

32. A user interface module, comprising a housing, logic, input interface, a communication system, and a component of an electrical connection.

33. A device for regulating cerebral spinal fluid drainage amounts from brain, spine, tissue or organs of a patient comprising a single module, further comprising a housing, logic, input interface, a sensor mechanism, a communication system to communicate a signal from the sensor mechanism, an energy source, a cerebral spinal fluid tubing pathway, and a valve mechanism, wherein the device is portable external gravity-based device.

34. The fluid-handling module for use with the device of claim 1, comprising a housing, a sensor mechanism, an energy source, a cerebral spinal fluid tubing pathway, a valve mechanism, and a component of an electrical connection.

35. The user interface module for use with the device of claim 1, comprising a housing, logic, an input interface, a communication system, and a component of an electrical connection.

36. The single module for use with the device of claim 1, comprising a housing, logic, an input interface, a sensor mechanism, a communication system to communicate a signal from the sensor mechanism, an energy source, a cerebral spinal fluid tubing pathway, and a valve mechanism.

37. The use of a device of the invention in a post neurosurgical or an ENT procedures wherein the post neurosurgical or the ENT procedure are triggered by the CSF encounter and wherein sealing of a dura mater at a surgical site via re-routing of the CSF from an alternate pathway in the form of an external ventricular drain or the lumbar drain is desired, wherein the dura mater is a sac which contains the brain, spinal cord and CSF.

38. The method of use of claim 37, wherein the post neurosurgical or the ENT procedure is a skull base surgery, a pituitary surgery, a traumatic skull base fractures, a post spinal surgery, a sinus surgery, wherein the sinus surgery is a ethmoid, sphenoid or mastoid.
